(19) United States
(12) Patent Application Publication
(10) Pub. No.: US 2005/0239780 A1
(43) Pub. Date: Oct. 27, 2005

(54) Tetrahydropyran derivative

(76) Inventors: Akira Suga, Tsukuba-shi (JP); Hideki Kubota, Tsukuba-shi (JP); Masanori Miura, Tsukuba-shi (JP); Daisuke Sasuga, Tsukuba-shi (JP); Hiroshi Moritani, Tsukuba-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/514,636

(22) PCT Filed: May 29, 2003
(86) PCT No.: PCT/JP03/06758

§ 371 Date: Nov. 17, 2004

(30) Foreign Application Priority Data
May 31, 2002 (JP) 2002-158555

(51) Int. Cl. A61K 31/5377; A61K 31/496; C07D 471/02

(52) U.S. Cl. 514/232.5; 544/125; 544/361; 514/253.03

(57) ABSTRACT

The invention has succeeded in settling the aforementioned problems by finding that a novel tetrahydropyran derivative has excellent apo B-related lipoprotein secretion-inhibiting activity.

### TECHNICAL FIELD

This invention relates to novel tetrahydropyran derivatives and their salts that have apo B-related lipoprotein secretion-inhibiting activity, to pharmaceutical compositions that comprise it as the active ingredient, and to combination with lipid lowering agents.

### BACKGROUND ART

Hyperlipemia is one risk factor of arteriosclerotic diseases such as ischemic heart disease, along with diabetes, hypertension, smoking and the like, and ameliorating it is effective in treatment of said diseases [JAMA. 1986; 256: 2835-0.2838, JAMA. 1986; 256: 2823-2828, JAMA. 1993; 269: 3015-3023]. Hypercholesterolemia is a risk factor of arteriosclerosis and causes coronary disorders, and hypertriglyceridemia is considered as one cause of ischemic heart disease such as myocardial infarction. Accordingly, for treatment of hyperlipemia, it is desirable to lower the level of cholesterol and triglyceride in blood. Of the remedies that have so far been in clinical use for hyperlipemia, HMG-CoA reductase inhibitors, anion-exchange resin compositions, probucol and like are essentially for lowering blood cholesterol, and fibrate and nicotinic acid compositions are essentially for lowering blood triglyceride.

Cholesterol absorbed in small intestine forms a chylomicron complex with apoprotein B (apo B), phospholipid and triglyceride, in the granular endoplasmic reticula of intestinal epithelial cells, and it is secreted into blood via lymph vessels and carried to tissues such as liver. Cholesterol synthesized in liver forms a VLDL (very-low-density lipoprotein) complex with apo B, phospholipid and triglyceride, in the granular endoplasmic reticula in liver cells, and it is secreted into blood, converted into LDL (low-density lipoprotein), and carried to other tissues [New England Journal of Medicine, 1983; 309: 288-296].

Apo B includes two molecular species, apo B-100 and apo B-48, and these are synthesized in the granular endoplasmic reticula of cells. In liver cells, synthesized is apo B-100, while in intestinal cells, synthesized is apo B-48 through apo B mRNA editing, and these are to be the structural apoproteins of VLDL and chylomicron, respectively. Cholesterol esters, triglycerides and others synthesized in the granular endoplasmic reticula are transferred by MTP, and bound to apo B in endoplasmic reticula to form a premature lipoprotein. The premature lipoprotein goes through the process of further lipid loading, sugar chain loading in Golgi body, and others to become a mature lipoprotein, which is then secreted outside cells [Biochem. Biophys. Acta., 1999; 1440: 1-31; Biochem. Biophys. Acta., 1997; 1345: 11-26].

Accordingly, when the secretion of apo B-related lipoprotein (generic term for chylomicron, VLDL and LDL) that comprises apo B as the constitutive component, from small intestine and/or liver into blood is inhibited, then it is possible to lower the level of cholesterol and triglyceride in blood, and such compounds are useful for remedies for hyperlipemia, arteriosclerosis, obesity, pancreatitis and the like.

For apo B-related lipoprotein production and/or secretion inhibitors, amide type compounds such as cycloalkano-indole and cycloalkano-azaindole derivatives disclosed in Patent Reference 1 or Patent Reference 2 have been reported. The compound described in Example 5 of Patent Reference 1 had been in clinical stages as Implitapide (Bay-13-9952). Apart from these, other various compounds including the hydrazide derivatives disclosed in Patent References 3 and 4 have been reported, but their efficacy is not satisfactory, and compounds having better and higher efficacy are desired.

On the other hand, compounds in which a nitrogen atom in a pyridoindole or carbazole ring is substituted with carbamoylmethyl-substituted benzyl have been reported by Patent References 1 and 2, but compounds in which a methyl moiety in said carbamoylmethyl is substituted with tetrahydropyran have not been reported yet.

[Patent Reference 1]

JP-A-8-225526
   [Patent Reference 2]
JP-A-10-45759
   [Patent Reference 3]
International Publication pamphlet WO 00/71502
   [Patent Reference 4]
International Publication pamphlet WO 01/74817

### DISCLOSURE OF THE INVENTION

An object of the invention is to provide novel tetrahydropyran derivatives and salts thereof which have excellent apo B-related lipoprotein secretion-inhibiting activity and are useful as agents for lowering cholesterol and triglyceride in blood, and further to provide medicaments that contain them.

With the aim of finding a novel compound having apo B-related lipoprotein secretion-inhibiting activity, the present inventors have conducted intensive studies and found a novel derivative having tetrahydropyran which has never known before. Thereafter, the invention has been accomplished by finding that said derivative having tetrahydropyran has strong apo B-related lipoprotein secretion-inhibiting activity and has strong cholesterol- and triglyceride-lowering activity.

That is, the invention relates to (1) a tetrahydropyran derivative represented by the following general formula (I) or a salt thereof 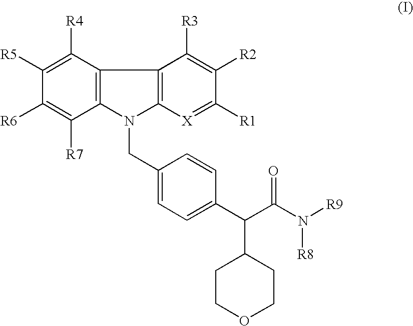
(symbols in the formula mean as follows;
R¹ and R³: the same or different and each represents H or lower alkyl,
R²: H, halogen, R^{a}-lower alkyl-, or R²⁰O—CO—,
R^{a}: H, R²¹O—CO—, R²²R²³N—, R²⁴R²⁵N—CO—, R²⁶O—, cyano, or optionally-substituted hetero ring-,
R⁴, R⁵, R⁶ and R⁷: the same or different and each represents H, halogen, haloalkyl, cyano, lower alkyl, lower alkyl-O—,
R²¹O—CO-lower alkyl, R²⁷—CO—, or R²⁸R²⁹N—S(O)₂—,
R⁸ and R⁹: the same or different and each represents H, lower alkyl, R³⁰-lower alkyl, R³¹R³²N—, optionally-substituted hetero ring-, or R³³R³⁴R³⁵C—, with the proviso that R⁸ and R⁹ may together form an optionally-substituted hetero ring-,
X: N, or CR³⁶,
R²⁰, R²² to R²⁶, R²⁸, R²⁹, R³², R³⁵ and R³⁶: the same or different and each represents H, or lower alkyl,
R²¹: H, lower alkyl, or aryl-lower alkyl-,
R²⁷: HO—, lower alkyl-O—, or optionally-substituted hetero ring-,
R³⁰: optionally-substituted aryl, optionally-substituted hetero ring-, or lower alkyl-O—,
R³³: HO-lower alkyl-, or optionally-substituted hetero ring-lower alkyl-, and
R³⁴: optionally-substituted aryl).

(2) The tetrahydropyran derivative or salt thereof described in (1), wherein the optionally-substituted aryl and optionally-substituted hetero ring in the aforementioned general formula (1) may be substituted with 1 to 3 substituents selected from the group shown below; substituents: halogen, oxo, lower alkyl optionally substituted with OH, haloalkyl-optionally substituted with lower alkyl-O—, lower alkenyl, C₃₋₈ cycloalkyl, C₃₋₈ cycloalkyl-lower alkyl-, aryl optionally substituted with halogen atom(s), aryl-lower alkyl-, heterocyclic group, —OH, lower alkyl-O—, lower alkyl-O-lower alkyl optionally substituted with OH, lower alkyl-O-lower alkyl-CO—, lower alkyl-S(O)ₘ—, lower alkyl-S(O)ₘ-lower alkyl-, lower alkyl-O—CO—, HO—CO-lower alkyl-, HO—CO-lower alkyl-O—, R^{x}R^{y}N—CO—, R^{x}R^{y}N—CO-lower alkyl-, R^{x}R^{y}N-lower alkyl-, lower alkyl-CO—, aryl-lower alkyl-O—CO— and hetero ring-O—CO—(symbols in the formulae means as follows;
R^{x} and R^{y}: the same or different and each represents H or lower alkyl,
m: 0, 1 or 2).

(3) The aforementioned tetrahydropyran derivative or salt thereof, wherein, in the aforementioned general formula (I), R² is R^{a}-lower alkyl-, R⁴, R⁵, R⁶ and R⁷ may be the same or different and each represents H or halogen, R⁸ and R⁹ may be the same or different and each represents H, lower alkyl or R³⁰-lower alkyl-, or R⁸ and R⁹ together form a optionally-substituted hetero ring, and X is N.

Particularly preferably, the aforementioned tetrahydropyran derivative or salt thereof, wherein R² is HO—CO-lower alkyl.

(4) A compound selected from 3-(2,4-dimethyl-9-{4-[(S)-2-oxo-2-(4-pyridin-2-ylpiperazin-1-yl)-1-(tetrahydro-2H-pyran-4-yl)ethyl]benzyl}-9H-pyrido[2,3-b]indol-3-yl)propanoic acid; 3-(2,4-dimethyl-9-{4-[(S)-2-oxo-2-(4-propylpiperazin-1-yl)-1-(tetrahydro-2H-pyran-4-yl)ethyl]benzyl}-9H-pyrido[2,3-b]indol-3-yl)propanoic acid; 3-(9-{4-[(S)-(benzylcarbamoyl)(tetrahydro-2H-pyran-4-yl)methyl]benzyl}-2,4-dimethyl-9H-pyrido[2,3-b]indol-3-yl)propanoic acid; 3-(2,4-dimethyl-9-{4-[(S)-(dimethylcarbamoyl)(tetrahydro-2H-pyran-4-yl)methyl]benzyl}-9H-pyrido[2,3-b]indol-3-yl)propanoic acid; 3-(2,4-dimethyl-9-{4-[(S)-2-morpholin-4-yl-2-oxo-1-(tetrahydro-2H-pyran-4-yl)ethyl]benzyl}-9H-pyrido[2,3-b]indol-3-yl)propanoic acid; 3-(2,4-dimethyl-9-{4-[(S)-2-(4-isobutylpiperazin-1-yl)-2-oxo-1-(tetrahydro-2H-pyran-4-yl)ethyl]benzyl}-9H-pyrido[2,3-b]indol-3-yl)propanoic acid; 3-9-{4-[(S)-2-(4-cyclobutylpiperazin-1-yl)-2-oxo-1-(tetrahydro-2H-pyran-4-yl)ethyl]benzyl}-2,4-dimethyl-9H-pyrido[2,3-b]indol-3-yl)propanoic acid; 3-9-{4-[(S)-2-(4-cyclopentylpiperazin-1-yl)-2-oxo-1-(tetrahydro-2H-pyran-4-yl)ethyl]benzyl}-2,4-dimethyl-9H-pyrido[2,3-b]indol-3-yl)propanoic acid; and their salts.

(5) A pharmaceutical composition which comprises the tetrahydropyran derivative described in the aforementioned (1) or a pharmaceutically acceptable salt thereof.

(6) A method for preventing or treating hyperlipemia, which comprises administering a therapeutically effective amount of the tetrahydropyran derivative of the aforementioned (1) or a pharmaceutically acceptable salt thereof and a pharmaceutically acceptable carrier to a patient who requires the treatment.

(7) A therapeutic agent for hyperlipemia, characterized in that the tetrahydropyran derivative described in the aforementioned (1) or a pharmaceutically acceptable salt thereof is administered in combination with one or two or more lipid lowering agents.

(8) The therapeutic agent described in the aforementioned (7), wherein the lipid lowering agent is selected from the group shown below;
(a) an HMG-CoA reductase inhibitor,
(b) acyl-CoA cholesterol acyltransferase (ACAT) inhibitor,
(c) a cholesterol absorption inhibitor,
(d) a nicotinic acid formulation,
(e) a fibrate drug,
(f) a bile acid adsorbent, and
(g) a medicament other than (a) to (f) which are used for the treatment of hyperlipemia.

(9) A method for preventing or treating hyperlipemia, by administering a therapeutically effective amount of the tetrahydropyran derivative described in the aforementioned (1) or a pharmaceutically acceptable salt thereof and a pharmaceutically acceptable carrier, simultaneously or separately with one or two or more lipid lowering agents, to a patient who requires the treatment.

(10) The therapeutic method described in the aforementioned (9), wherein the lipid lowering agent is selected from the group shown below;
(a) an HMG-CoA reductase inhibitor,
(b) acyl-CoA cholesterol acyltransferase (ACAT) inhibitor,
(c) a cholesterol absorption inhibitor,
(d) a nicotinic acid formulation,
(e) a fibrate system medicament,
(f) a bile acid adsorbent, and
(g) a medicament other than (a) to (f) which are used for the treatment of hyperlipemia.

(11) A kit in which a pharmaceutical composition containing the tetrahydropyran derivative described in the aforementioned (1) or a pharmaceutically acceptable salt thereof and a pharmaceutically acceptable carrier and a lipid lowering agent are each independently packaged.

### MODE FOR CARRYING OUT THE INVENTION

The following further describes the invention.

Further description on the compound represented by the general formula (I) is as follows.

Unless otherwise noted, the term “lower” as used herein in defining the general formula means a linear or branched carbon chain having from 1 to 6 carbon atoms.

“Lower alkyl” is C₁₋₆ alkyl, preferably C₁₋₄ alkyl such as methyl, ethyl, propyl, isopropyl or t-butyl, more preferably C₁₋₃ alkyl.

“Aryl” is a mono- to tricyclic aromatic hydrocarbon ring group which is from 6- to 14-membered as a whole, and is preferably phenyl, naphthyl or anthranyl.

“Halogen” includes, for example, fluorine, chlorine, bromine and iodine atoms.

“Haloalkyl” is a group of the aforementioned lower alkyl of which any hydrogen atom(s) is substituted with the aforementioned halogen, and is preferably trifluoromethyl.

“Hetero ring” means a “saturated hetero ring” and an “unsaturated hetero ring” having from 1 to 3 hetero atoms selected from nitrogen atom, oxygen atom and sulfur atom, which may be condensed.

“Unsaturated hetero ring” means 5- or 6-membered unsaturated heterocyclic group having from 1 to 3 hetero atoms selected from nitrogen atom, oxygen atom and sulfur atom, which may be condensed, and includes aromatic hetero rings. Preferably, it is a 5- or 6-membered monocyclic unsaturated hetero ring containing 1 or 2 nitrogen atoms, including pyrrole, 3-pyrroline, 3,6-dihydropyrimidine, 3,6-dihydropyridine, pyridine, furan, thiazole, thiophene, imidazole, triazole, tetrazole, pyrimidine and pyridazine.

“Saturated hetero ring” means 3- or 10-membered saturated hetero ring having from 1 to 3 hetero atoms selected from nitrogen atom, oxygen atom and sulfur atom, which may be condensed. Spiro-structured rings and bridged rings are also included in said saturated hetero ring. Illustratively, it includes azetidine, pyrrolidine, piperidine, piperazine, azepine, diazepine, morpholine, thiomorpholine, thiazolidine, 1,4-dioxa-8-azaspiro[4,5]decane, isoindoline, dihydroisoindole, tetrahydroisoquinoline, 2,5-diazabicyclo[2.2.1]heptane and tetrahydropyran. Preferably, it is a 4- to 7-membered saturated hetero ring.

“Optionally-substituted hetero ring formed by R⁸ and R⁹ together” means a hetero ring of those mentioned above, which has at least one nitrogen atom as the ring-forming hetero atom. Preferably, it is a 5- or 6-membered saturated or unsaturated hetero ring having one or two nitrogen atoms as ring-forming hetero atoms, and these hetero rings may be substituted with lower alkyl, lower alkylene, aryl optionally substituted with halogen, cycloalkyl or pyridyl, and may be condensed with benzene ring.

Optionally-substituted aryl and hetero ring may have from 1 to 3 substituents.

The substituents mean those which are generally used in the field of the substituted groups, and their examples include halogen, oxo, lower alkyl optionally substituted with OH, haloalkyl-optionally substituted with lower alkyl-O—, lower alkenyl, lower alkynyl, C₃₋₈ cycloalkyl, C₃₋₈ cycloalkyl-lower alkyl-, aryl optionally substituted with halogen atom(s), aryl-lower alkyl-, heterocyclic group, hetero ring-lower alkyl-, HO—, lower alkyl-O—, lower alkyl-O-lower alkyl-optionally substituted with OH, lower alkyl-O-lower alkyl-CO—, lower alkenyl-O—, lower alkynyl-O—, C₃₋₈ cycloalkyl-O—, aryl-O—, aryl-lower alkyl-O—, hetero ring-lower alkyl-O—, lower alkyl-S(O)ₘ— (m: 0, 1 or 2; the same shall apply hereinafter), lower alkyl-S(O)ₘ-lower alkyl-, lower alkenyl-S(O)ₘ— lower alkynyl-S(O)ₘ—, C₃₋₈ cycloalkyl-S(O)ₘ—, aryl-S(O°)ₘ—, aryl-lower alkyl-S(O)ₘ—, hetero ring-S(O)ₘ—, hetero ring-lower alkyl-S(O)ₘ—, lower alkyl-O—CO—, lower alkenyl-O—CO—, lower alkynyl-O—CO—, C₃₋₈ cycloalkyl-O—CO—, aryl-O—CO—, aryl-lower alkyl-O—CO—, hetero ring-O—CO—, hetero ring-lower alkyl-O—CO—, —COOH, HOOC-lower alkyl-, HOOC-lower alkyl-O—, lower alkyl-CONR^{x}— (R^{x} is the same or different and each means H or lower alkyl; the same shall apply hereinafter), lower alkyl-CONR^{x}-lower alkyl-, R^{x}R^{y}N— (RY is the same or different and each means H or lower alkyl; the same shall apply hereinafter), R^{x}R^{y}N-lower alkyl-, R^{x}R^{y}N—CO—, R^{x}R^{y}N—CO-lower alkyl-, HCO—, lower alkyl-CO—, lower alkenyl-CO—, lower alkynyl-CO—, C₃₋₈ cycloalkyl-CO—, aryl-CO—, aryl-lower alkyl-CO—, hetero ring-CO—, hetero ring-lower alkyl-CO—, HCS—, lower alkyl-CS—, lower alkenyl-CS—, lower alkynyl-CS—, C₃₋₈ cycloalkyl-CS—, aryl-CS—, aryl-lower alkyl-CS—, hetero ring-CS—, hetero ring-lower alkyl-CS—, lower alkyl-O—CO—CO—, HCO—O—, lower alkyl-CO—O—, lower alkenyl-CO—O—, lower alkynyl-CO—O—, C₃₋₈ cycloalkyl-CO—O—, aryl-CO—O—, aryl-lower alkyl-CO—O—, hetero ring-CO—O—, hetero ring-lower alkyl-CO—O—, aryl-lower alkyl-O—CO— and hetero ring-O—CO—.

Preferred are halogen, oxo, lower alkyl optionally substituted with OH, haloalkyl-optionally substituted with lower alkyl-O—, lower alkenyl, C₃₋₈ cycloalkyl, C₃₋₈ cycloalkyl-lower alkyl-, aryl optionally substituted with halogen, aryl-lower alkyl-, heterocyclic group, —OH, lower alkyl-O—, lower alkyl-O-lower alkyl-optionally substituted with OH, lower alkyl-O-lower alkyl-CO—, lower alkyl-S(O)ₘ—, lower alkyl-S(O)ₘ-lower alkyl-, lower alkyl-O—CO—, HOOC-lower alkyl-, HOOC-lower alkyl-O—, R^{x}R^{y}N—CO—, R^{x}R^{y}N—CO-lower alkyl-, R^{x}R^{y}N-lower alkyl-, lower alkyl-CO—, aryl-lower alkyl-O—CO— and hetero ring-O—CO—.

Illustrative examples of the “C₃₋₈ cycloalkyl” include cyclopropyl, cyclobutyl, cyclopentyl and cyclohexyl. Preferably, it is cycloalkyl having from 3 to 6 carbon atoms, particularly preferably from 4 to 6 carbon atoms.

Especially preferred as the compound of the invention is the tetrahydropyran derivative or a salt thereof according to the first embodiment of the invention, wherein, in the aforementioned general formula (I), R² is COOH-lower alkyl-, R⁴, R⁵, R⁶ and R⁷ are the same or different, each representing H or halogen, R⁸ and R⁹ are the same or different, each representing H or aryl optionally substituted with halogen-lower alkyl-, or R⁸ and R⁹ may together form hetero ring having 1 or 2 nitrogen atoms as ring atoms, and X is N.

The compounds (I) of the invention have an asymmetric carbon, and include (R)- and (S)-optical isomers and racemic bodies based on it. Further, depending on the type of the substituents therein, the compounds may have multiple asymmetric carbons, and include diastereomers and enantiomers based on these. The invention encompasses all such isomers isolated and their mixtures.

The compounds (I) of the invention may form salts with acids or bases. Such salts include acid-addition salts with mineral acids such as hydrochloric acid, hydrobromic acid, hydroiodic acid, sulfuric acid, nitric acid and phosphoric acid, or with organic acids such as formic acid, acetic acid, propionic acid, oxalic acid, malonic acid, succinic acid, fumaric acid, maleic acid, lactic acid, malic acid, citric acid, tartaric acid, carbonic acid, picric acid, methanesulfonic acid, ethanesulfonic acid and glutamic acid.

The salts with bases include salts with inorganic bases such as sodium, potassium, magnesium, calcium and aluminum, salts with organic bases such as methylamine, ethylamine, meglumine, ethanolamine, diethanolamine and tromethamine, salts with basic amino acids such as lysine, arginine and ornithine, and ammonium salts.

The invention further encompasses hydrates, solvates with ethanol or the like, and polymorphic crystals of the compounds (I) of the invention.

The compounds of the invention further include pharmaceutically acceptable prodrugs. The groups that form the pharmaceutically acceptable prodrugs of the compounds of the invention are describe, for example, in Prog. Med. 5: 2157-2161 (1985) and “Development of Medicines” published in 1990 by Hirokawa Shoten, vol. 7, Molecular Planning, pp. 163-198. Illustratively, they are groups capable of being converted into primary amines, secondary amines, OH, COOH and the like of the invention through hydrolysis or solvolysis or under physiological condition, and their examples as prodrugs of OH group include —OC(O)-[lower alkyl which may be substituted]-C(O)OR (R represents H or a lower alkyl, the same shall apply hereinafter), —OC(O)-[lower alkenylene which may be substituted]-C(O)OR, —OC(O)-[aryl which may be substituted], —OC(O)-[lower alkyl]-O-[lower alkyl]-C(O)OR, —OC(O)—C(O)R, —OC(O)-[a lower alkyl which may have be substituted], —OSO₂-[a lower alkyl which be substituted]-C(O)OR, —O-phthalidyl, 5-methyl-1,3-dioxolen-2-on-4-yl-methyloxy or the like.

The following agents can be exemplified as the lipid lowering agent to be used in combination with the invention.
(a) An HMG-CoA reductase inhibitor,
(b) acyl-CoA cholesterol acyltransferase (ACAT) inhibitor,
(c) a cholesterol absorption inhibitor,
(d) a nicotinic acid preparation,
(e) a fibrate system medicament,
(f) a bile acid adsorbent, and
(g) a medicament other than (a) to (f) which are used for the treatment of hyperlipemia.

Illustrative examples of respective agents are described in the following.

Examples of the (a) an HMG-CoA reductase inhibitor include pravastatin, simvastatin, fluvastatin, lovastatin, cerivastatin, atorvastatin, pitavastatin and the like.

Examples of the (b) acyl-CoA cholesterol acyltransferase (ACAT) inhibitor include melinalide.

Examples of the (c) a cholesterol absorption inhibitor include cholestyramine, colestipol and the like anion exchange resins.

Examples of the (d) a nicotinic acid formulation include niceritrol, nicomol, tocopherol nicotinate and the like.

Examples of the (e) a fibrate system medicament include clofibrate, clinofibrate, simfibrate, bezafibrate, gemfibrozil and the like.

Examples of the (f) a bile acid adsorbent include abalelix and the like.

Examples of the (g) a medicament other than (a) to (f) which are used for the treatment of hyperlipemia include probucol, phosphatidylcholine, riboflavin butyric acid ester, elastase, ethyl eicosapetaenoate and dextran sulfate sodium, as well as polyene phosphatidylcholine, soysterol, gamma oryzanol, pantethine, ursodeoxycholic acid and the like.

The compounds (I) and their pharmaceutically acceptable salts have blood lipid lowering effect based on their apo B-related lipoprotein secretion-inhibiting activity, and are useful for remedies for hyperlipemia, arteriosclerosis, obesity and pancreatitis. Of the compounds of the invention, those with R² of HO—CO-lower alkyl- have weak inhibitory action upon chemical metabolism enzyme cytochrome P450 (CYP), especially CYP3A4, and therefore have few side effects, so that these are especially useful as medicaments for the aforementioned diseases. The CYP3A4-inhibiting activity can be determined by the same method described in WO 02/44179, page 16, lines 9 to 22.

Production Methods

The compounds (I) of the invention can be produced according to any of the following first to sixth methods, depending on the type of the substituents therein. In case where the compounds of the invention are optically active compounds, the compounds (I) of the invention can be synthesized by properly selecting the reaction conditions mentioned below, with no problem of racemation during their production, using the optically active compounds (XVII*) mentioned below.
First Method

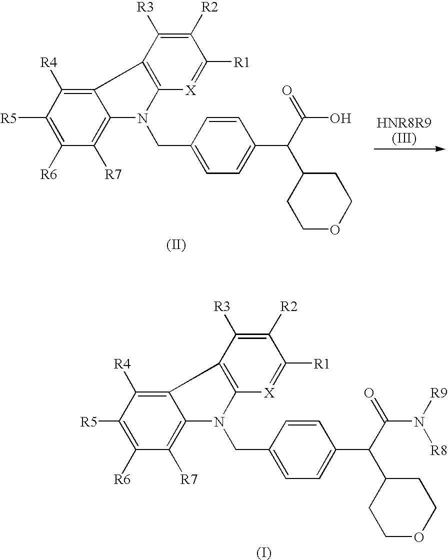

The compounds (I) of the invention can be synthesized through condensation of a carboxylic acid compound represented by the compound (II) with an amine compound represented by the compound (III). The reaction is carried out in an organic solvent generally not having any influence on the reaction, in the presence of a condensing agent, at room temperature or under heat. The solvent includes methylene chloride, chloroform, 1,2-dichloroethane, dimethylformamide, dimethylacetamide, 1-methylpyrrolidinone, tetrahydrofuran and the like. The condensing agent includes, for example, dicyclohexylcarbodiimide (DCC), 1-ethyl-3-(3-dimethylaminopropyl)carbodiimide hydrochloride (WSC HCl), diphenylphosphorylazide (DPPA), carbonyldiimidazole (CDI), diisopropylcarbodiimide (DIPCI) and the like. The reaction may be carried out in the presence of a base, and examples of the base include triethylamine, diisopropylethylamine, 4-methylmorpholine, 1,8-diazabicyclo[5,4,0]-7-undecene (DBU) and the like. The reaction may also be carried out in the presence of an activator, and examples of the activator include 1-hydroxybenzotriazole (HOBt), hydroxysuccinimide (HOSu), dimethylaminopyridine (DMAP), hydroxyphthalimide (HOPht) and the like. Preferably, the compounds (II) and (III) are used in an equimolar ratio or one of them in excessive in some degree, and the condensing agent and the base or the activator to be used is 1 to 2 equivalents to the compound (II) or (III).

Not through the reaction with the aforementioned condensing agent, the compound (I) may also be synthesized by converting the compound (II) into an acid chloride which can be derived through its reaction with thionyl chloride or oxalyl chloride, or a mixed acid anhydride that can be derived through its reaction with ethyl chloroformate or isopropyl chloroformate, and then allowing the resulting reactive derivative to react with the compound (III) in the presence of the base mentioned above.
Second Method

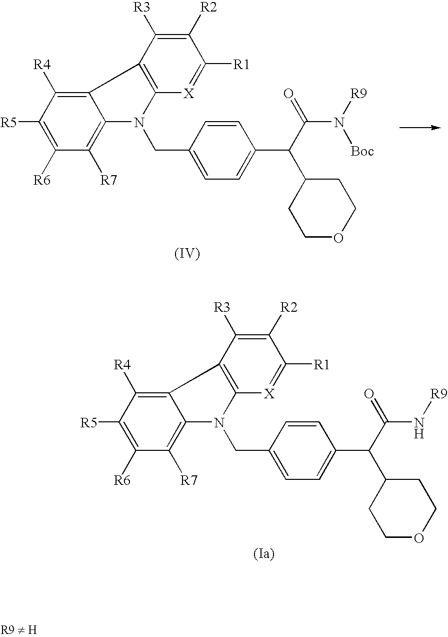

Of the compounds of the invention, secondary amide compounds (Ia) can be synthesized through deprotection of a compound (IV) of which the amido moiety is protected with a tert-butoxycarbonyl group (Boc group). The reaction can be carried out in an organic solvent generally not having any influence on the reaction, such as dichloromethane, chloroform or dichloroethane, in the presence of an acid such as hydrochloric acid or trifluoroacetic acid, at room temperature or under heat.

Some of the compounds (I) of the invention can be synthesized by further converting the substituents in the compounds obtained through the aforementioned first or second method into the intended substituents. The substituent conversion may be effected in any desired manner depending on the type of the intended substituents, for example, according to any of the following third to sixth methods.
Third Method

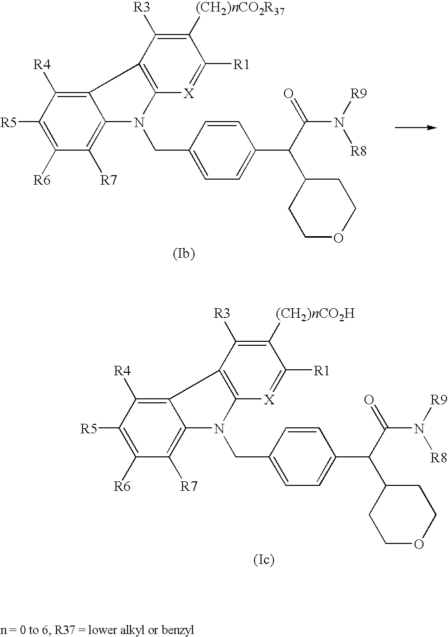

Of the compounds of the invention, carboxylic acid compounds (Ic) can be synthesized through hydrolysis of the ester compounds (Ib) obtainable in the first method. In case where R37 is a lower alkyl group, the reaction is carried out in a mixed solvent of an organic solvent generally not having any influence on the reaction, such as methanol, ethanol, tetrahydrofuran or dioxane, and water, in the presence of a base or an acid, which is used in the same or excessive amount, at room temperature or under heat. As the base, an inorganic base such as sodium hydroxide, potassium hydroxide or potassium carbonate is used, and hydrochloric acid, sulfuric acid, hydrobromic acid or the like is used as the acid. In case where R37 is benzyl group, the carboxylic acid compounds (Ic) can be obtained through hydrolysis of benzyl esters (Ib). Preferably, the reaction is carried out in an organic solvent generally not having any influence on the reaction, such as ethanol, ethyl acetate, tetrahydrofuran, dimethylformamide or acetic acid, in a hydrogen gas atmosphere in the presence of a metal catalyst such as palladium-carbon, at room temperature.
Fourth Method

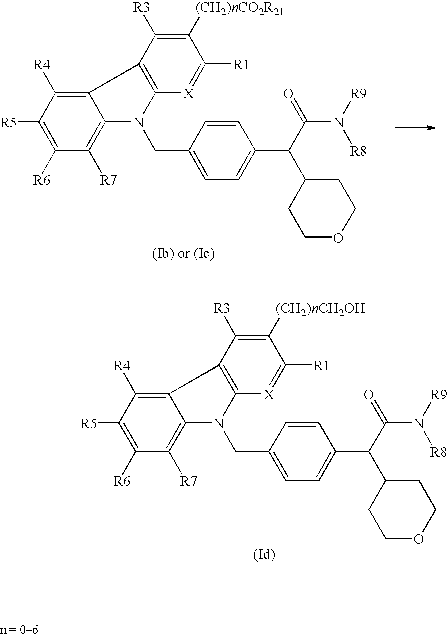

Of the compounds of the invention, alcohol compounds (Id) can be synthesized through reduction of the ester compounds (Ib) obtainable in the first method or the carboxylic acid compounds (Ic) obtainable in the second method. The reaction can be carried out in an organic solvent generally not having any influence on the reaction, in the presence of a suitable reducing agent. The solvent includes tetrahydrofuran, diethyl ether, dioxane and dimethoxyethane. The suitable reducing agent includes borane-tetrahydrofuran complex, lithium aluminum hydride and lithium borohydride. Preferably, the amount of the reducing agent to be used is 1 to 2 equivalents to the compound (Ib) or (Ic).

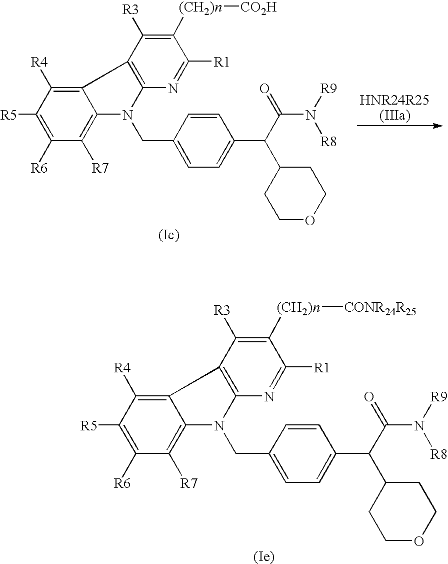

Of the compounds of the invention, amide compound (Ie) can be synthesized through condensation of the compound (Ic) obtainable in the second method with an amine compound (IIIa). The reaction can be carried out in the same manner as in the first method.
Sixth Method

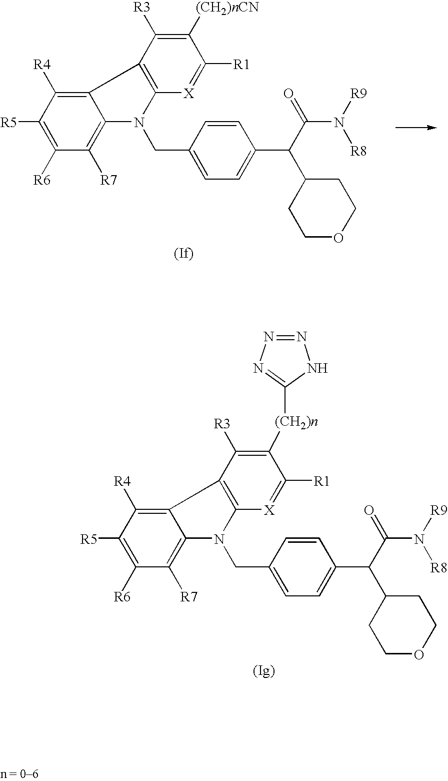

Of the compounds of the invention, tetrazole compounds (Ig) can be synthesized by allowing the nitrile compound (If) obtainable in the first method to react with an azidating agent. The reaction can be carried out in an organic solvent generally not having any influence on the reaction such as toluene, xylene or benzene, under heat. For the azidating agent, preferred are tributyltin azide and sodium azide.

A method for preparing carboxylic acid compounds (II) to be used as the starting compounds in the aforementioned first production method is described below. The carboxylic acid compounds (II) can be synthesized from a compound (V) and a bromide compound (VI) through the steps 1 and 2 mentioned below.

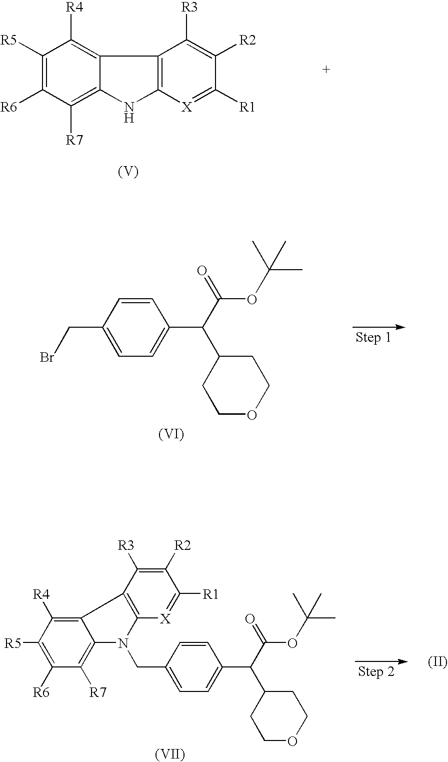

That is, the carboxylic acid compounds (II) can be synthesized through ordinary hydrolysis of tert-butyl ester compounds (VII) under an acidic condition usual for those skilled in the art. The reaction can be carried out using hydrochloric acid in an organic solvent not having any influence on the reaction such as dioxane or tetrahydrofuran, or using trifluoroacetic acid in an organic solvent such as dichloromethane, chloroform or 1,2-dichloroethane, at room temperature or under heat.

The tert-butyl ester compounds (VII) can be synthesized by allowing a compound (V), which is obtainable in the method mentioned below, to react with a bromide compound (VI) in the presence of a base. Preferably, the compound (V) is reacted with the compound (VI) in an equimolar ratio or one of the two is excessive in some degree over the other, in an organic solvent generally not having any influence on the reaction, such as dimethylformamide, dimethylacetamide, 1-methylpyrrolidinone or tetrahydrofuran, in the presence of a base that is equimolar to or excessive in some degree over the compound (V), with cooling or at room temperature. For the base, preferred are potassium tert-butoxide and sodium hydride.

Next, methods for preparing the aforementioned starting compounds (V) and the bromide compounds (VI) are described below in that order.

Firstly, the compounds (V) can be synthesized from known 2-halogenonitrobenzene compounds (VIII) through the following steps 3 to 6. 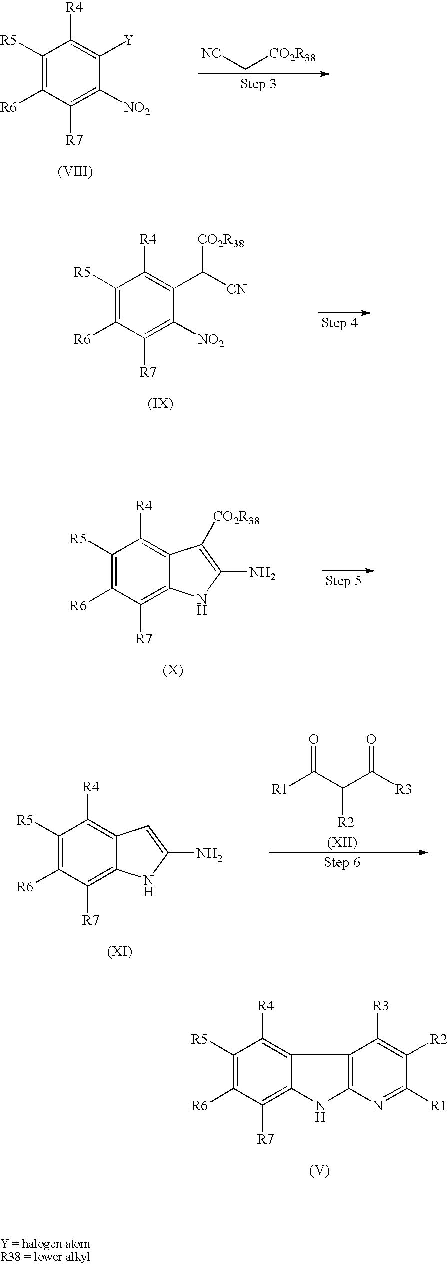
Step 3

Compounds (IX) can be synthesized by allowing a compound (VIII) to react with a cyanoacetate (preferably ethyl cyanoacetate or tert-butyl cyanoacetate) in the presence of a base, in accordance with the method described in C. A. Grob, O. Weissbach, Helv. Chim. Acta., 44, 1748 (1961). For the base, preferred are potassium tert-butoxide and sodium hydride. Te reaction is carried out in an organic solvent generally not having any influence on the reaction, such as pyridine, dimethylformamide, dimethylacetamide or 1-methylpyrrolidinone, under heat, in which the cyanoacetate and the base may be two equivalents to the compound (VIII).

Step 4

Compounds (X) can be synthesized by allowing the compound (IX) to react with a reducing agent, in accordance with the method described in K. L. Munshi, H. Kohl, N. J. de Souza, J. Heterocyclic Chem., 14, 1145 (1977). For the reducing agent, preferred are zinc, iron and tin(II) chloride. Preferably, the reaction is carried out in an acidic solvent such as acetic acid or hydrochloric acid, under heat at 80° C. or higher.

Step 5

Compounds (XI) can be synthesized through hydrolysis followed by decarboxylation of the ester compound (X) in an acidic condition, in accordance with the method described in R. A. Glennnon, J. Heterocyclic Chem., 12, 135 (1975). Preferably, the reaction is carried out in an acidic solvent such as hydrochloric acid, sulfuric acid or trifluoroacetic acid, under heat for reflux.

Step 6

Compounds (V) can be synthesized by allowing the compound (XI) to react with a compound (XII) in the presence of a base, in accordance with the method described in R. S. Sagitullin, T. V. Mel'nikova, A. N. Kost, V. F. Snegirev, E. N. Frenkel, Chemistry of Heterocyclic Compounds (English translation), 9, 968 (1973). The reaction is carried out in an organic solvent generally not having any influence on the reaction, under heat. As the base, triethylamine, potassium hydroxide or the like is used, and ethanol or isopropyl alcohol is desirable as the solvent.

Some compounds among the compounds (V) can be synthesized by further converting the substituents in the compounds (V) obtained through the steps 3 to 6, into the intended substituents. Such a substituent conversion can be carried out in any desired manner depending on the type of the intended substituents, for example, according to the following process.

That is, compounds (Vb), (Vc) and (Vd) can be synthesized from the compounds (Va) obtainable through the steps 3 to 6, according to the following steps 7-1,7-2 and 7-3 in that order. 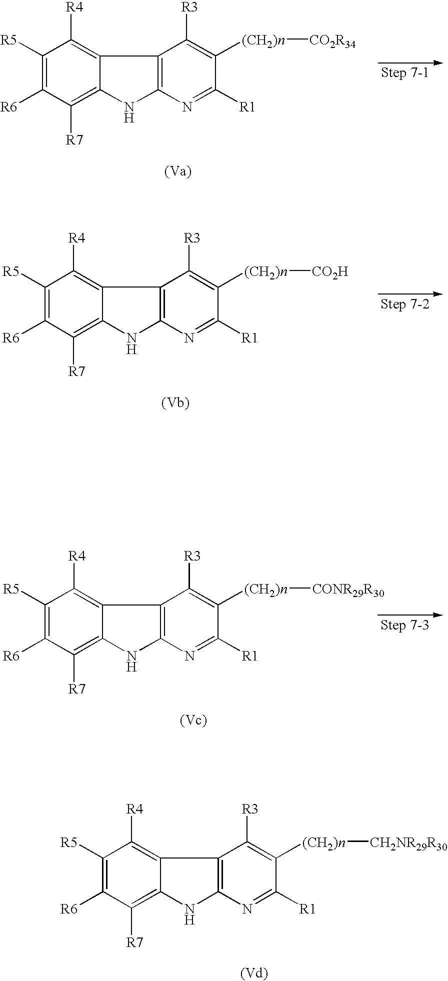
Step 7-1

Compounds (Vb) can be synthesized through hydrolysis of the compounds (Va) with an ordinary acid or base usual for those skilled in the art, and the reaction can be carried out in the same manner as in the third production method.

Step 7-2

Compounds (Vc) can be synthesized through amidation of the compounds (Vb) with an ordinary condensing agent usual for those skilled in the art, and the reaction can be carried out in the same manner as in the first production method.

Step 7-3

Compounds (Vd) can be synthesized through reduction of the compounds (Vc). The reaction can be carried out in an organic solvent generally not having any influence on the reaction, in the presence of a suitable reducing agent at room temperature or under heat. The solvent includes tetrahydrofuran, diethyl ether, dioxane and dimethoxyethane. The suitable reducing agent includes lithium aluminum hydride, lithium borohydride and diborane-tetrahydrofuran complex. Preferably, the amount of the reducing agent to be used is 1 to 2 equivalents to the compound (Vc).

Compounds (Ve) and (Vf) can be synthesized from the aforementioned compounds (Vb) through the following steps 7-4 and 7-5 in that order. 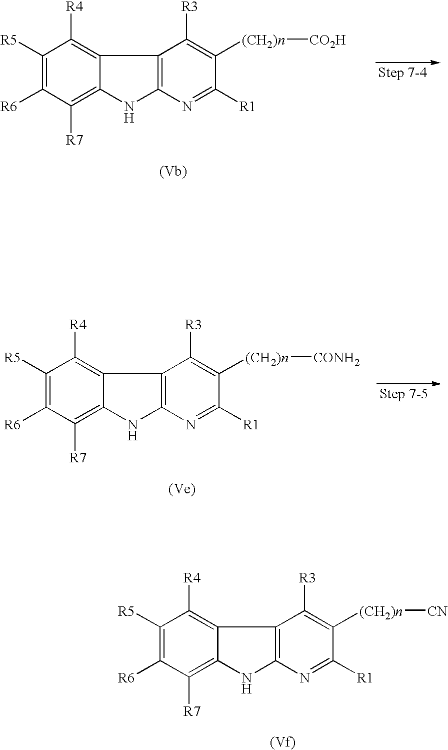
Step 7-4

Compounds (Ve) can be synthesized through amidation of the compounds (Vb) with ammonium chloride, ammonium carbonate, aqueous ammonia, ammonia gas or the like and with an ordinary condensing agent usual for those skilled in the art, and the reaction can be carried out in the same manner as in the first production method.

Step 7-5

Compounds (Vf) can be synthesized through dehydration of the compounds (Ve). Preferably, the reaction is carried out in an organic solvent generally not having any influence on the reaction, in the presence of a dehydrating agent such as phosphorus oxychloride, with cooling.

On the other hand, the bromide compounds (VI) can be synthesized from known compounds (XIII) through the following steps 8 to 13. 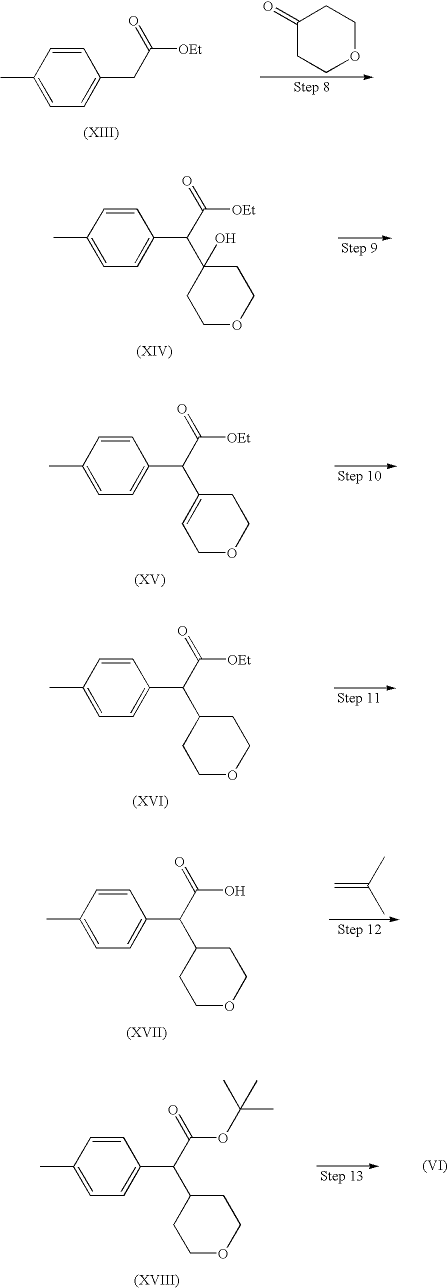
Step 8

Compounds (XIV) can be synthesized by allowing a compound (XIII) to react with tetrahydro-2H-pyran-4-one. The reaction is carried out in an organic solvent generally not having any influence on the reaction, in the presence of a base with cooling at −78° C. to −20° C. For the base, preferred are organometal amides such as lithium hexamethyldisilazide and lithium diisopropylamide, and preferred as the solvent are tetrahydrofuran and diethyl ether.

Step 9

Compounds (XV) can be synthesized through dehydration of the compounds (XIV) under an acidic condition. Preferably, the reaction is carried out in an organic solvent not having any influence on the reaction such as ethanol or dioxane, or in water, in the presence of an acid such as sulfuric acid or hydrochloric acid, under heat at 40 to 80° C.

Step 10

Compounds (XVI) can be synthesized through catalytic reduction of the compounds (XV). Preferably, the reaction is carried out in an organic solvent generally not having any influence on the reaction such as ethanol, ethyl acetate, tetrahydrofuran or acetic acid, in a hydrogen gas atmosphere in the presence of a metal catalyst such as palladium-carbon, at room temperature.

Step 11

Compounds (XVII) can be synthesized through hydrolysis of the compounds (XVI). The reaction is carried out in a mixed solvent of an organic solvent generally not having any influence on the reaction, such as methanol, ethanol or tetrahydrofuran, and water, in the presence of a base or an acid under heat. For the base, preferred are inorganic bases such as sodium hydroxide and potassium hydroxide, and preferred as the acid are hydrochloric acid, sulfuric acid and hydrobromic acid.

Step 12

Compounds (XVIII) can be synthesized through tert-butylation of the compounds (XVII). The reaction is carried out in an organic solvent generally not having any influence on the reaction, such as dichloroethane, dichloromethane or chloroform, at room temperature in the presence of isobutene gas (this is dissolved in the solvent) and by the use of a catalytically effective amount of sulfuric acid. Alternatively, the compounds (XVII) may be reacted with isobutene gas, which is formed in the system by the use of an excessive amount of tert-butyl alcohol, in the presence of an excessive amount of anhydrous magnesium sulfate and an equimolar amount of sulfuric acid at room temperature to give the compound, in accordance with the method described in S. T. Wright, D. L. Hageman, A. S. Wright, L. D. McClure, Tetrahedron Letters, 38 (42), 7345 (1997).

Step 13

The compounds (VI) can be synthesized through bromination of the compounds (XVIII). The reaction is carried out in an organic solvent generally not having any influence on the reaction, such as carbon tetrachloride or ethyl acetate, by the use of a brominating agent such as N-bromosuccinimide, in the presence of a catalytically effective amount of a radical initiator such as N,N′-azobisisobutyronitrile or benzoyl peroxide, under heat for reflux or with exposure to light.

On the other hand, optically pure compounds (XVII*) can be synthesized through optical resolution of the aforementioned racemic compounds (XVII) by the following steps 14 and 15. 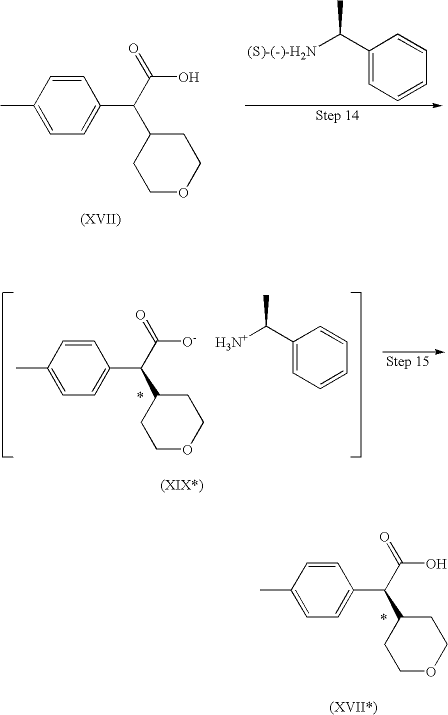
Step 14

Compounds (XIX*) that are pure to the level of diastereomers can be synthesized through differential recrystallization of salts formed by reacting the compound (XVII) with (S)-(−)-1-phenylethylamine, which shall be repeated once or multiple times. The reaction of the compound (XVII) with (S)-(−)-1-phenylethylamine is carried out in an organic solvent generally not having any influence on the reaction such as tetrahydrofuran or acetonitrile, at room temperature or under heat, and it is desirable to use from 0.5 to 1 equivalent of (S)-(−)-1-phenylethylamine relative to the racemic compound (XVII), and after the recrystallization, it is desirable that the salt is left at room temperature for a few hours and then taken out trough filtration. It is desirable that the subsequent differential crystallization of the (S)-(−)-1-phenylethylammonium salt is carried out in a solvent of tetrahydrofuran.

Step 15

The optically pure compounds (XVII*) can be synthesized by carrying out desalting of the compounds (XIX*) that are pure to the level of diastereomers. The desalting is carried out in a two-layer system of an organic solvent such as ethyl acetate or chloroform, and water, at room temperature by the use of an equimolar or excessive amount of an acid such as hydrochloric acid or sulfuric acid.

The optically pure compounds (XVII*) can be led to optically pure compounds (I*) of the invention through a process that comprises the aforementioned step 12, step 13, step 1 and step 2 and the first production method in that order.

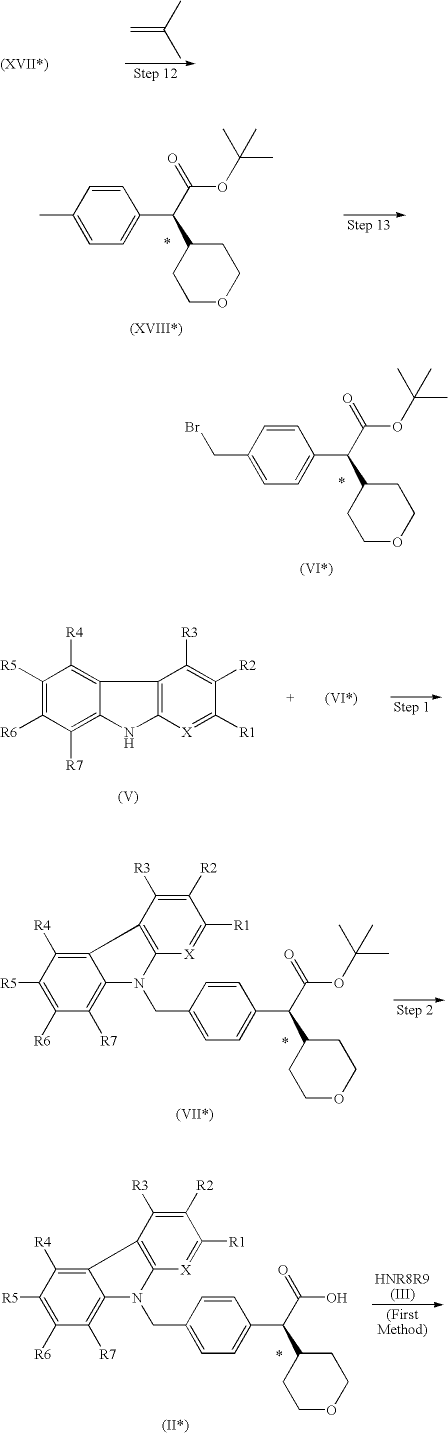

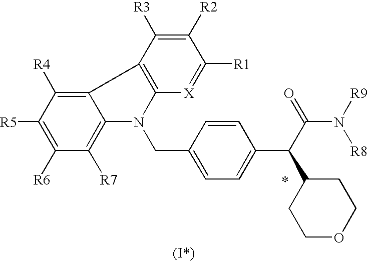

A method for preparing Boc-protected amide compounds (IV), which are used as the starting compounds in the aforementioned second production method, is described below. 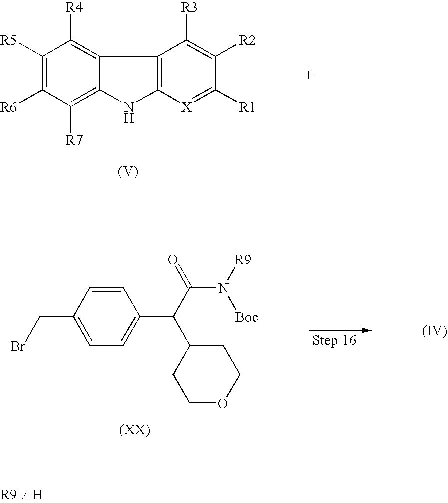
Step 16

Compounds (IV) can be synthesized from the aforementioned compound (V) and a bromide compound (XX) which is obtainable by the method mentioned below, in the presence of a base in the same manner as in the step 1. Preferably, the compound (V) is allowed to react with a compound (XX) in an equimolar ratio or one of the two is excessive in some degree in an organic solvent generally not having any influence on the reaction, such as dimethylformamide, dimethylacetamide, 1-methylpyrrolidinone or tetrahydrofuran, in the presence of a base which is equimolar to or excessive over the compound (V), with cooling or at room temperature. For the base, preferred are potassium tert-butoxide and sodium hydride.

Next, a method for preparing the aforementioned bromide compounds (XX) is described.

The compounds (XX) can be synthesized from the aforementioned carboxylic acid compounds (XVII) through the steps 17 to 19 shown below. 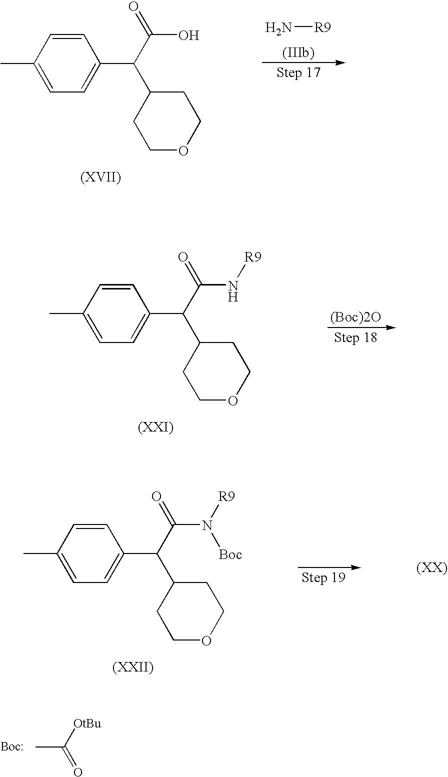
Step 17

Secondary amide compounds (XXI) can be synthesized through condensation of the carboxylic acid compound (XVII) with an amine compound (IIIb), and the reaction can be carried out in the same manner as in the first production method.

Step 18

Compounds (XXII) can be synthesized by allowing the compound (XXI) to react with di-tert-butyl dicarbonate (DIBOC). The reaction can be carried out by allowing the compound (XXI) to reacted with an equimolar or slightly excessive amount of DIBOC in an organic solvent generally not having any influence on the reaction, such as acetonitrile, tetrahydrofuran or dioxane, in the presence of a catalytically effective amount of 4-dimethylaminopyridine (DMAP) at room temperature.

Step 19

The bromide compounds (XX) can be synthesized through bromination of the compounds (XXII), and the reaction can be carried out in the same manner as in the aforementioned step 13.

In case where an optically pure compound (XVII*) is used in the step 17, then it can be led to optically pure compounds (Ia*) of the invention through the steps 18, 19 and 16 and the second production method.

The compounds of the invention are isolated and purified as free compounds, their salts, hydrates, solvates or polymorphic crystals. Pharmaceutically acceptable salts of the compounds (I) of the invention can be produced through ordinary salt formation.

Isolation and purification can be carried out by any of ordinary chemical operations such as extraction, differential crystallization and various modes of partitioning chromatography.

Isomers can be separated from each other by selecting suitable starting compounds, or by utilizing the difference in the physical properties between the isomers. For example, optical isomers can be led to stereochemically pure isomers by selecting suitable starting materials or through racemic isolation of racemic compounds (e.g., by leading to a diastereomer salt with an ordinary optically active base followed by its optical resolution).

The pharmaceutical preparations that contain, as the active ingredient, one or two more of the compounds and their salts of the invention are prepared by the use of a carrier, a filler and other additives generally used in formulating pharmaceutical compositions.

They may be orally administered in any form of tablets, pills, capsules, granules, powders or liquids, or may be parenterally administered in any form of injections such as intravenous injections or intramuscular injections, or suppositories or subcutaneous preparations. Their dose may be suitably determined, depending on the symptoms, the age and the sex of the patients to which they are administered, but is, in general, from 0.01 to 500 mg/adult/day for oral administration, and from 0.001 to 100 mg/adult/day for parenteral administration, and this may be administered all at a time or by dividing into a few portions in 2 to 4 times.

Regarding the lipid lowering agent which is jointly used with the tetrahydropyran derivative of the invention, an appropriate amount is selected within the effective amount of said lipid lowering agent.

The lipid lowering agent to be used jointly with the tetrahydropyran derivative of the invention may be made into a medical mixture, or the aforementioned two or more agents may be separately made into pharmaceutical preparations in advance and separately administered. Its therapeutic effects are sufficiently expressed by any of these administration methods.

As the solid composition for oral administration of the compounds of the invention, employed are tablets, powders, granules and the like. In the solid composition of such types, one or more active substances are mixed with at least one inert diluent, such as lactose, mannitol, glucose, hydroxypropylcellulose, microcrystalline cellulose, starch, polyvinyl pyrrolidone or magnesium metasilicate aluminate. In an ordinary manner, the composition may contain other additives in addition to the inert diluents, including a lubricant such as magnesium stearate, a disintegrator such as calcium cellulose glycolate, a stabilizer such as lactose, and a dissolution promoter such as glutamic acid or aspartic acid. As occasion demands, the tablets or pills may be coated with a film of sugar or gastric or enteric substances such as sucrose, gelatin, hydroxypropylcellulose and hydroxypropylmethyl cellulose phthalate.

The liquid composition for oral administration includes pharmaceutically acceptable emulsions, solutions, suspensions, syrups, elixirs and the like, which contain ordinary inactive diluents such as purified water or ethanol. In addition to the inert diluents, such compositions may further contain wetting promoters, suspension promoters and the like assisting agents, as well as sweeteners, flavorings, aromas and preservatives.

Injection is an example of the typical administration method of parenteral administration, and he injections include aseptic aqueous or non-aqueous solutions, suspensions and emulsions. The diluent for the aqueous solutions and suspensions includes, for example, distilled water and physiological saline for injections. The diluent for the non-aqueous solutions and suspensions includes, for example, propylene glycol, polyethylene glycol, vegetable oils such as olive oil, alcohols such as ethanol, and polysorbate 80 (trade name). Such compositions may further contain additives such as preservatives, wetting promoters, emulsifiers, dispersants, stabilizers (e.g., lactose) and dissolution promoters (e.g., glutamic acid and aspartic acid). These are sterilized by filtering them through bacteria-trapping filters, or by adding bactericides thereto, or by exposing them to radiations. In addition, sterile solid compositions may be produced in advance, and they may be dissolved in sterile water or in a sterile solvent for injection, before using them.

### EXAMPLES

The invention is described further in detail with reference to the following Examples. The invention, however, is not restricted by these Examples. In this connection, methods for producing the starting compounds used in the Examples are described as Reference Examples.

### Reference Example 1

### Ethyl (4-hydroxytetrahydro-2H-pyran-4-yl)(4-methylphenyl)acetate

A 550 ml portion of a solution of 1 M lithium bis(trimethylsilyl)amide in tetrahydrofuran (THF) was cooled to −70° C. in a stream of argon, 50 ml of a THF solution of 92.5 g of ethyl (4-methylphenyl)acetate was added dropwise thereto spending 30 minutes, and then stirred at the same temperature for 30 minutes. Under cooling at −70° C., 50 ml of a THF solution of 52.3 g of tetrahydro-2H-pyran-4-one was added dropwise to this reaction liquid spending 30 minutes and then stirred at the same temperature for 1 hour. The reaction liquid was mixed with 1,000 ml of 10% citric acid aqueous solution and then extracted with 500 ml of ethyl acetate, and the organic layer was washed with 500 ml of 10% citric acid aqueous solution and 500 ml of saturated brine in that order. After drying with anhydrous magnesium sulfate, the solvent was evaporated under a reduced pressure to obtain pale yellow crystals. By washing the crystals with 400 ml of n-hexane, 145.7 g of the title compound was obtained as colorless crystals.

FAB-MS m/z: 279 (M⁺+1)

### Reference Example 2

### Ethyl 3,6-dihydro-2H-pyran-4-yl(4-methylphenyl)acetate

A 300 ml portion of concentrated sulfuric acid was gradually added dropwise to 300 ml of ethanol, and then cooled to 10° C. in an ice bath. A 145 g portion of ethyl (4-hydroxytetrahydro-2H-pyran-4-yl)(4-methylphenyl)acetate synthesized in Reference Example 1 was added to this solution, and then stirred at 60° C. for 3 hours. To the reaction liquid were added 500 ml of ethyl acetate and 1,500 ml of water, and then the water layer was further extracted with ethyl acetate. Both of the ethyl acetate layers were combined, washed with 500 ml of saturated brine and then dried with anhydrous magnesium sulfate. The solvent was evaporated under a reduced pressure, and then the residue was applied to a silica gel column chromatography and eluted with ethyl acetate/n-hexane (1/9, v/v) to obtain 92.0 g of the title compound as a pale yellow oil.

FAB-MS m/z: 261 (M⁺+1)

### Reference Example 3

### Ethyl (4-methylphenyl)(tetrahydro-2H-pyran-4-yl)acetate

A 92.0 g portion of ethyl (3,6-dihydro-2H-pyran-4-yl)(4-methylphenyl)acetate synthesized in Reference Example 2 was dissolved in 800 ml of ethanol, and 10.2 g of 10% Pd—C was added thereto in an Ar atmosphere. The reactor was purged with hydrogen gas, and then the matter therein was vigorously stirred at room temperature for 4 hours. The reactor was again purged with Ar gas, and then the insoluble matter was removed by filtration and the solvent was evaporated under a reduced pressure to obtain 89.9 g of the title compound as a pale yellow oil.

FAB-MS m/z: 263 (M⁺+1)

### Reference Example 4

### (4-Methylphenyl)(tetrahydro-2H-pyran-4-yl)acetic acid

A 89.7 g portion of ethyl (4-methylphenyl)(tetrahydro-2H-pyran-4-yl)acetate synthesized in Reference Example 3 was dissolved in 300 ml of ethanol, and then 170 ml of 8 M sodium hydroxide aqueous solution was added thereto and heated under reflux for 4.5 hours. After evaporation of ethanol under a reduced pressure, the residue was mixed with 120 ml of concentrated hydrochloric acid and extracted with 500 ml of ethyl acetate. The organic layer was washed with 200 ml of saturated brine and dried with anhydrous magnesium sulfate, and then the solvent was evaporated under a reduced pressure to obtain a colorless oil. This was dried under a reduced pressure and crystallized to obtain 74.5 g of the title compound as colorless crystals.

FAB-MS m/z: 233 (M⁺+1)

### Reference Example 5

### (2S)-(4-methylphenyl)(tetrahydro-2H-pyran-4-yl)acetic acid

(4-Methylphenyl)(tetrahydro-2H-pyran-4-yl)acetic acid synthesized in Reference Example 4 was optically resolved by the following method. That is, 74.1 g of (±)-2-(4-methylphenyl)-2-(tetrahydro-2H-pyran-4-yl)acetic acid was dissolved in 700 ml of acetonitrile at 80° C. After addition of 16.0 g of triethylamine, 19.2 g of (S)-(−)-phenylethylamine was gradually added dropwise thereto to find formation of colorless crystals. This was cooled to room temperature and then stirred at the same temperature for 2 hours. The thus formed crystals were collected by filtration, washed with 100 ml of acetonitrile and then dried to obtain 55.0 g of colorless crystals. The crystals were mixed with 2,600 ml of THF and dissolved therein by heating under reflux. About 300 ml of THF was evaporated by heating, and then the residue was cooled to room temperature to give colorless crystals. They were stirred overnight at the same temperature. The thus formed crystals were collected by filtration, washed with 500 ml of THF and then dried to obtain 43.6 g of (S)-(−)-phenylethylamine salt of the title compound as colorless crystals. This was mixed with 700 ml of ethyl acetate and 500 ml of 1 M hydrochloric acid and vigorously stirred at room temperature for 1 hour. The ethyl acetate layer was washed with saturated brine and dried with anhydrous magnesium sulfate, and then the solvent was evaporated under a reduced pressure to obtain 29.0 g of the title compound as colorless crystals.

[α]25, D=48.3 (c=1.00, CHCl₃)

### Reference Example 6

### tert-Butyl (2S)-(4-methylphenyl)(tetrahydro-2H-pyran-4-yl)acetate

A 64.5 g portion of (2S)-(4-methylphenyl)(tetrahydro-2H-pyran-4-yl)acetic acid synthesized in Reference Example 5 was dissolved in 900 ml of methylene chloride, and 2.5 ml of concentrated sulfuric acid was added thereto. While cooling the reaction liquid to −70° C., isobutene gas was introduced into the reaction liquid to liquefy it, about 500 ml of the resulting liquid was pooled, and this solution was stirred overnight at room temperature. (When necessary, 500 ml of isobutene gas was again added thereto with cooling, and further stirred overnight at room temperature.) After evaporation of the solvent under a reduced pressure, the residue was mixed with 1,000 ml of chloroform and washed with 1,000 ml of saturated sodium bicarbonate aqueous solution and 1,000 ml of saturated brine in that order. The organic layer was dried with anhydrous magnesium sulfate, and then the solvent was evaporated under a reduced pressure to obtain 70.5 g of the title compound as a colorless solid.

FAB-MS m/z: 291 (M⁺+1), [α]25, D=17.7 (c=1.00, CHCl₃)

### Reference Example 7

### tert-Butyl (2S)-(4-bromomethylphenyl)(tetrahydro-2H-pyran-4-yl)acetate

A 46.5 g portion of tert-butyl (2S)-(4-methylphenyl)(tetrahydro-2H-pyran-4-yl)acetate synthesized in Reference Example 6 was dissolved in 600 ml of carbon tetrachloride, 1.3 g of 2,2′-azobisisobutyronitrile (AIBN) and 34.2 g of N-bromosuccinimide (NBS) were added thereto in that order at 50° C., and then the reaction liquid was heated under reflux for 1 hour. After ice-cooling of the reaction liquid, the insoluble matter was removed by filtration, and then the solvent was evaporated under a reduced pressure. The residue was applied to a silica gel column chromatography and eluted with ethyl acetate/n-hexane (1/20→1/10, v/v) to obtain 34.5 g of the title compound as colorless crystals.

¹H NMR (CDCl₃) δ: 1.08-1.18 (2H, m), 1.38-1.41 (10H, m), 2.08-2.25 (1H, m), 3.14 (1H, d), 3.22-3.33 (1H, m), 3.42 (1H, dt), 3.80-3.90 (1H, m), 3.93-4.03 (1H, m), 4.48 (2H, s), 7.29 (2H, d), 7.34 (2H, d) [α]25, D=8.9 (c=1.00, CHCl₃)

### Reference Example 8

### N-benzyl-2-(4-methylphenyl)-2-(tetrahydro-2H-pyran-4-yl)acetamide

A 8.6 ml portion of benzylamine was dissolved in 150 ml of DMF, and 15.4 g of (4-methylphenyl)(tetrahydro-2H-pyran-4-yl)acetic acid synthesized in Reference Example 4, 15.1 g of 1-ethyl-3-(3-dimethylaminopropyl) carbodiimide hydrochloride (WSC HCl) and 10.7 g of 1-hydroxybenzotriazole (HOBt) were added thereto in that order at room temperature and stirred overnight at the same temperature. After evaporation of the reaction liquid under a reduced pressure, the residue was mixed with ethyl acetate and saturated sodium bicarbonate aqueous solution and extracted with ethyl acetate. The organic layer was washed with water, 1.0 M hydrochloric acid and saturated brine and dried with anhydrous magnesium sulfate, and then the solvent was evaporated under a reduced pressure. The residue was recrystallized from a mixed solvent of diethyl ether and h-hexane to obtain 17.6 g of the title compound as colorless crystals.

FAB-MS m/z: 324 (M⁺+1)

### Reference Example 9

### tert-Butyl N-benzyl-[(4-methylphenyl)(tetrahydro-2H-pyran-4-yl)]ethanoylcarbamate

A 15.6 g portion of N-benzyl-2-(4-methylphenyl)-2-(tetrahydro-2H-pyran-4-yl)acetamide was dissolved in 160 ml of acetonitrile, and 589 mg of 4-dimethylaminopyridine (DMAP) and 12.6 g of di-tert-butyl dicarbonate (Boc2O) were added thereto in that order at room temperature and stirred overnight at the same temperature. Ethyl acetate and distilled water were added to the reaction liquid. The organic layer was washed with saturated brine and dried with anhydrous magnesium sulfate, and then the solvent was evaporated under a reduced pressure. The residue was applied to a silica gel column chromatography and eluted with a mixed solvent of n-hexane/ethyl acetate (4/1, v/v) to obtain 16.8 g of the title compound as a colorless oil.

FAB-MS m/z: 424 (M⁺+1)

### Reference Example 10

### tert-Butyl N-benzyl-[(4-methylphenyl)(tetrahydro-2H-pyran-4-yl)]ethanoylcarbamate

A 16.8 g portion of (O)-tert-butyl N-benzyl-[2-(4-methylphenyl)-2-(tetrahydro-2H-pyran-4-yl)]ethanoylcarbamate synthesized in Reference Example 9 was dissolved in 170 ml of carbon tetrachloride, 326 mg of 2,2′-azobisisobutyronitrile (AIBN) and 7.42 g of N-bromosuccinimide (NBS) were added thereto in that order at 60° C., and then the reaction liquid was heated under reflux for 1 hour. After evaporation of the reaction liquid under a reduced pressure, the residue was applied to a silica gel column chromatography and eluted with ethyl acetate/n-hexane (1/15→1/9, v/v) to obtain 13.8 g of the title compound as colorless foam substance.

FAB-MS m/z: 402 (M⁽⁺⁾−Boc), ¹H NMR (DMSO) δ: 1.31 (9H, s)

### Reference Example 11

### 4-Chloro-3-nitro-benzenesulfonyl chloride

A 26.2 g portion of sodium 4-chloro-3-nitro-benzenesulfonate was dissolved in 250 ml of 1,2-dichloroethane, and 26.2 g of thionyl chloride and 4.0 g of DMF were added thereto and heated under reflux for 6 hours. The solvent was evaporated under a reduced pressure, the residue was mixed with 400 ml of ethyl acetate, washed with saturated sodium bicarbonate aqueous solution and saturated brine in that order and dried with anhydrous magnesium sulfate, and then the solvent was evaporated under a reduced pressure to obtain 12.3 g of the title compound as a colorless oil.

¹H NMR (CDCl₃) δ: 7.88 (1H, d), 8.18 (1H, dd), 8.54 (1H, d)

### Reference Example 12

### 4-Chloro-N,N-dimethyl-3-nitro-benzenesulfonamide

A 7.7 g portion of 4-chloro-3-nitro-benzenesulfonyl chloride synthesized in Reference Example 11 was dissolved in 80 ml of 1,2-dichloroethane, and 2.7 g of dimethylamine hydrochloride and 7.4 g of triethylamine were added thereto under ice-cooling and stirred at room temperature for 2 hours. The reaction liquid was washed with 1 M hydrochloric acid, saturated sodium bicarbonate aqueous solution and saturated brine in that order and then dried with anhydrous magnesium sulfate. After evaporation of the solvent under a reduced pressure, the residue was applied to a silica gel column chromatography and eluted with chloroform to obtain 4.12 g of the title compound as pale yellow solid.

FAB-MS m/z: 264 (M⁺+1)

### Reference Example 13

### Ethyl cyano-[4-(N,N-dimethylaminosulfonyl)-2-nitrophenyl]acetate

A 3.5 g portion of potassium tert-butoxide was dissolved in 50 ml of pyridine, and 3.5 g of ethyl cyanoacetate and a pyridine solution (15 ml) of 4.1 g of 4-chloro-N,N-dimethyl-3-nitro-benzenesulfonamide synthesized in Reference Example 12 were added thereto in that order and stirred at 80° C. for 30 minutes. The solvent was evaporated under a reduced pressure, and then 150 ml of toluene and 200 ml of water were added thereto. The water layer was acidified with 1 M hydrochloric acid and extracted with 200 ml of ethyl acetate, and then the organic layer was washed with saturated brine. After drying with anhydrous magnesium sulfate, the solvent was evaporated under a reduced pressure to obtain 5.76 g of the title compound as a red oil.

FAB-MS m/z: 342 (M⁺+1)

### Reference Example 14

### Ethyl 2-amino-6-(N,N-dimethylaminosulfonyl)-1H-indole-3-carboxylate

A 9.2 g portion of ethyl cyano-[4-(N,N-dimethylaminosulfonyl)-2-nitrophenyl]acetate synthesized in Reference Example 13 was dissolved in 100 ml of acetic acid, and 4.5 g of reduced iron was added thereto at 100° C. After reacted vigorously, this was stirred at 100° C. for 1 hour. The reaction liquid was cooled to room temperature, and the insoluble matter was removed by filtration. The residue was mixed with 200 ml of ethyl acetate and 100 ml of 1 M hydrochloride, the organic layer was washed with 1 M hydrochloric acid and saturated brine in that order and dried with anhydrous magnesium sulfate, and ten the solvent was evaporated under a reduced pressure. The residue was mixed with 60 ml of diisopropyl ether and heated under reflux for 20 minutes. After cooling to room temperature, the insoluble matter was collected by filtration and dried to obtain 4.83 g of the title compound as a bluish purple solid.

FAB-MS m/z: 312 (M⁺+1)

### Reference Example 15

### tert-Butyl 2-amino-6-cyano-1H-indole-3-carboxylate

A 18.4 g portion of potassium tert-butoxide was dissolved in 150 ml of pyridine, and 17 ml of tert-butyl cyanoacetate and 15.0 g of 1-chloro-5-cyano-2-nitrobenzene were added thereto in that order and stirred at 80° C. for 4 hours. The solvent was evaporated under a reduced pressure, and then the residue was mixed with 200 ml of water and washed with 300 ml of toluene. The water layer was mixed with 120 ml of concentrated hydrochloric acid and then extracted with 300 ml of ethyl acetate. The organic layer was washed with saturated brine and dried with anhydrous magnesium sulfate, and then the solvent was evaporated under a reduced pressure to obtain 22.9 g of crude tert-butyl (±)-cyano-(4-cyano-2-nitrophenyl)acetate as a dark red oil. This was used in the next reaction without further purification.

A 22.6 g portion of the crude tert-butyl (±)-cyano-(4-cyano-2-nitrophenyl)acetate was dissolved in 200 ml of acetic acid and heated to 100° C. This was mixed with 13.2 g of reduced iron and stirred at 100° C. for 1.5 hours. This was cooled to room temperature, the insoluble matter was removed by filtration, and then the solvent was evaporated under a reduced pressure. The residue was mixed with 300 ml of ethyl acetate and 300 ml of 1 M hydrochloric acid, and the reaction solution was filtered through Celite. The filtrate was extracted with ethyl acetate, and then the organic layer was washed with 1 M hydrochloric acid and saturated brine and dried with anhydrous magnesium sulfate. The solvent was evaporated under a reduced pressure, and the residue was applied to a silica gel column chromatography and eluted with ethyl acetate/n-hexane (3/7, v/v) to obtain 7.94 g of the title compound as a dark brown foam.

FAB-MS m/z: 258 (M⁺+1)

### Reference Example 16

### 6-(N,N-dimethylaminosulfonyl)-2-imino-2,3-dihydro-1H-indole monohydrochloride

A 20 ml portion of concentrated hydrochloric acid was added to 4.83 g of ethyl 2-amino-6-(N,N-dimethylaminosulfonyl)-1H-indole-3-carboxylate, and this was heated under reflux for 1 hour. The solvent was evaporated under a reduced pressure, 20 ml of ethanol was added to the residue to dissolve it at 80° C., and then this was returned to room temperature. The solid precipitated by adding 20 ml of diethyl ether was collected by filtration and dried to obtain 3.14 g of the title compound as a pale brown solid.

FAB-MS m/z: 240 (M⁺+1)

### Reference Example 17

### 6-Cyano-2-imino-2,3-dihydro-1H-indole monohydrochloride

A 3.00 g portion of tert-butyl 2-amino-6-cyano-1H-indole-3-carboxylate synthesized in Reference Example 15 was dissolved in 30 ml of 1,2-dichloroethane, mixed with 15 ml of trifluoroacetate at room temperature and stirred overnight. After evaporation of the solvent under a reduced pressure, the residue was dissolved in 30 ml of ethyl acetate, and 4 ml of a solution of 4 M hydrogen chloride in ethyl acetate was added thereto. The thus formed solid was collected by filtration and dried to obtain 1.84 g of the title compound as a gray solid.

FAB-MS m/z: 158 (M⁺+1)

### Reference Example 18

### Ethyl 3-(2,4-dimethyl-9H-pyrido[2,3-b]indol-3-yl)propanoate

A 80 ml portion of isopropyl alcohol was added to 8.0 g of 2-imino-2,3-dihydro-1H-indole monohydrochloride, and 9.5 g of ethyl 4-acetyl-5-oxohexanoate and 14.4 g of triethylamine were added thereto in that order and stirred overnight at 80° C. The reaction liquid was cooled to room temperature, and the formed crystals were collected by filtration. A 90 ml portion of methanol was added thereto, this was heated under reflux directly as the suspended state and then cooled to room temperature, and the crystals were collected by filtration and dried to obtain 7.75 g of the title compound as gray crystals.

In the same manner as in Reference Example 18, the compounds shown in Table 1 below were synthesized. The meanings of the abbreviations in the table are described below.

Rex: Reference Example DATA: physicochemical properties NMR: proton nuclear magnetic resonance spectrum (TMS internal standard) δ, DMSO-d₆ unless otherwise specifically noted

TABLE 1

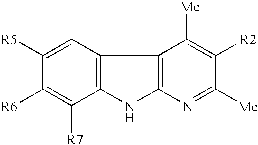

| Rex | R2 | R5 | R6 | R7 | DATA |
|---|---|---|---|---|---|
| 18 | (CH2)2CO2Et | H | H | H | FAB-MS |
| | | | | | mz:297(M⁺ + 1) |
| 19 | H | Cl | CF3 | H | FAB-MS |
| | | | | | mz:299(M⁺ + 1) |
| 20 | H | H | CO2H | H | FAB-MS |
| | | | | | mz:241(M⁺ + 1) |
| 21 | (CH2)3CH3 | H | H | H | FAB-MS |
| | | | | | mz:253(M⁺ + 1) |
| 22 | CH2COMe | H | H | H | FAB-MS |
| | | | | | mz:269(M⁺ + 1) |
| 23 | (CH2)2CO2Et | F | H | H | FAB-MS |
| | | | | | mz:315(M⁺ + 1) |
| 24 | (CH2)2CO2Et | H | F | H | FAB-MS |
| | | | | | mz:315(M⁺ + 1) |
| 25 | (CH2)2CO2Et | H | H | F | FAB-MS |
| | | | | | mz:315(M⁺ + 1) |
| 26 | (CH2)2CO2Et | Cl | H | H | FAB-MS |
| | | | | | mz:331(M⁺ + 1) |
| 27 | (CH2)2CO2Et | Me | H | H | FAB-MS |
| | | | | | mz:311(M⁺ + 1) |
| 28 | (CH2)2CO2H | MeO | H | H | FAB-MS |
| | | | | | mz:327(M⁺ + 1) |
| 29 | (CH2)2CO2Et | Cl | CF3 | H | FAB-MS |
| | | | | | mz:399(M⁺ + 1) |
| 30 | (CH2)2CO2Et | H | CN | H | FAB-MS |
| | | | | | mz:322(M⁺ + 1) |
| 31 | (CH2)2CO2Et | H | CO2H | H | FAB-MS |
| | | | | | mz:341(M⁺ + 1) |
| 32 | (CH2)2CO2Et | H | SO2NMe2 | H | FAB-MS |
| | | | | | mz:404(M⁺ + 1) |
| 33 | (CH2)2CO2CH2Ph | H | H | H | FAB-MS |
| | | | | | mz:359(M⁺ + 1) |
| 34 | (CH2)3CO2Et | H | H | H | FAB-MS |
| | | | | | mz:311(M⁺ + 1) |

### Reference Example 35

### Methyl 3-(2,4-dimethyl-9H-pyrido[2,3-b]indol-3-yl)-7-carboxylate

A 1.19 g portion of 3-(2,4-dimethyl-9H-pyrido[2,3-b]indol-3-yl)-7-carboxylic acid synthesized in Reference Example 20 was dissolved in 50 ml of methanol, mixed with 0.5 ml of concentrated sulfuric acid and heated overnight under reflux. After evaporation of the solvent under a reduced pressure, the residue was mixed with 100 ml of chloroform and washed with saturated sodium bicarbonate aqueous solution and saturated brine in that order. The organic layer was dried with anhydrous magnesium sulfate, and then the solvent was evaporated under a reduced pressure to obtain 930 mg of the title compound as a brown solid.

FAB-MS m/z: 255 (M⁺+1)

### Reference Example 36

### Methyl 3-(2,4-dimethyl-7-methoxycarbonyl-9H-pyrido[2,3-b]indol-3-yl)propanoate

A 1.0 g portion of ethyl 3-(7-carboxy-2,4-dimethyl-9H-pyrido[2,3-b]indol-3-yl)propanoate synthesized in Reference Example 31 was dissolved in 20 ml of methanol, mixed with 0.5 ml of concentrated sulfuric acid and stirred overnight. After evaporation of the solvent under a reduced pressure, the residue was mixed with 100 ml of chloroform and washed with saturated sodium bicarbonate aqueous solution and saturated brine in that order. The organic layer was dried with anhydrous magnesium sulfate, and then the solvent was evaporated under a reduced pressure to obtain 840 mg of the title compound as a colorless solid.

FAB-MS m/z: 341 (M⁺+1)

### Reference Example 37

### (2,4-Dimethyl-9H-pyrido[2,3-b]indol-3-yl)acetic acid

A 870 mg portion of methyl (2,4-dimethyl-9H-pyrido[2,3-b]indol-3-yl)acetate synthesized in Reference Example 22 was dissolved in 16 ml of methanol, mixed with 7 ml of 1 M sodium hydroxide aqueous solution and heated under reflux for 19 hours. Aqueous citric acid was added thereto, and the formed solid was collected by filtration and dried to obtain 760 mg of the title compound as a colorless solid.

FAB-MS m/z: 255 (M⁺+1)

### Reference Example 38

### (2,4-Dimethyl-9H-pyrido[2,3-b]indol-3-yl)-N,N-dimethylacetamide

A 400 mg portion of (2,4-dimethyl-9H-pyrido[2,3-b]indol-3-yl)acetic acid synthesized in Reference Example 37 was dissolved in 8 ml of DMF, 256 mg of dimethylamine hydrochloride, 0.66 ml of triethylamine, 361 mg of 1-ethyl-3-(3-dimethylaminopropyl)carbodiimide hydrochloride (WSC HCl) and 255 mg of 1-hydroxybenzotriazole (HOBt) were added thereto in that order at room temperature, and this was stirred overnight at the same temperature. Sodium bicarbonate aqueous solution was added to the reaction solution, and the formed solid was collected by filtration and dried to obtain 320 mg of the title compound as a colorless solid.

FAB-MS m/z: 282 (M⁺+1)

### Reference Example 39

### 2-(2,4-Dimethyl-9H-pyrido[2,3-b]indol-3-yl)-1-(morpholin-4-yl)ethanone

A 360 mg portion of (2,4-dimethyl-9H-pyrido[2,3-b]indol-3-yl)acetic acid synthesized in Reference Example 37 was dissolved in 8 ml of DMF, 0.25 g of morpholine, 361 mg of 1-ethyl-3-(3-dimethylaminopropyl)carbodiimide hydrochloride (WSC HCl) and 255 mg of 1-hydroxybenzotriazole (HOBt) were added thereto in that order at room temperature, and this was stirred overnight at the same temperature. Sodium bicarbonate aqueous solution was added to the reaction solution, and the formed solid was collected by filtration and dried to obtain 350 mg of the title compound as a colorless solid.

FAB-MS m/z: 324 (M⁺+1)

### Reference Example 40

### 2,4-Dimethyl-3-[2-(N,N-dimethylamino)ethyl]-9H-pyrido[2,3-b]indole

A 320 mg portion of (2,4-dimethyl-9H-pyrido[2,3-b]indol-3-yl)-N,N-dimethylacetamide synthesized in Reference Example 38 was added to a THF solution of 75 mg of lithium aluminum hydride and heated under reflux for 3.5 hours. A 120 mg portion of lithium aluminum hydride was added to the reaction solution and heated under reflux for 4 hours. Distilled water, 1 M sodium hydroxide aqueous solution and ethyl acetate were added to the reaction liquid in that order, and this was filtered through Celite. The reaction solution was extracted with ethyl acetate and washed with saturated brine, and then the organic layer was dried with anhydrous magnesium sulfate. By evaporating the solvent under a reduced pressure, 288 mg of the title compound was obtained as a pale yellow solid.

FAB-MS m/z: 268 (M⁺+1)

### Reference Example 41

### 2,4-Dimethyl-3-[2-(morpholin-4-yl)ethyl]-9H-pyrido[2,3-b]indole

A 350 mg portion of 2-(2,4-dimethyl-9H-pyrido[2,3-b]indol-3-yl)-1-(morpholin-4-yl)ethanone was added to a THF solution of 82 mg of lithium aluminum hydride and heated under reflux for 9 hours. Distilled water, 1 M sodium hydroxide aqueous solution and ethyl acetate were added to the reaction liquid in that order, and this was filtered through Celite. The reaction solution was extracted with ethyl acetate and washed with saturated brine, and then the organic layer was dried with anhydrous magnesium sulfate. By evaporating the solvent under a reduced pressure, 338 mg of the title compound was obtained as a pale yellow solid.

FAB-MS m/z: 310 (M⁺+1)

### Reference Example 42

### 3-(2,4-Dimethyl-9H-pyrido[2,3-b]indol-3-yl)propanoic acid

A 2.40 g portion of ethyl 3-(2,4-dimethyl-9H-pyrido[2,3-b]indol-3-yl)propanoate synthesized in Reference Example 18 was dissolved in 30 ml of ethanol, and this was mixed with 16 ml of 1 M sodium hydroxide aqueous solution and heated overnight under reflux. Aqueous citric acid was added thereto, and the formed solid was collected by filtration and dried to obtain 2.17 g of the title compound as a colorless solid.

FAB-MS m/z: 269 (M⁺+1)

### Reference Example 43

### 3-(2,4-Dimethyl-9H-pyrido[2,3-b]indol-3-yl)propanamide

A 268 mg portion of 3-(2,4-dimethyl-9H-pyrido[2,3-b]indol-3-yl)propanoic acid synthesized in Reference Example 42 was dissolved in 4 ml of DMF, 288 mg of 1-ethyl-3-(3-dimethylaminopropyl)carbodiimide hydrochloride (WSC HCl) and 203 mg of 1-hydroxybenzotriazole (HOBt) were added thereto in that order at room temperature, and this was stirred at the same temperature for 1.5 hours. The reaction liquid was mixed with 160 mg of ammonium chloride and 0.70 ml of diisopropylethylamine and stirred overnight. Sodium bicarbonate aqueous solution was added to the reaction solution, and the formed solid was collected by filtration and dried to obtain 230 mg of the title compound as a colorless solid.

FAB-MS m/z: 268 (M⁺+1)

### Reference Example 44

### 3-(2,4-Dimethyl-9H-pyrido[2,3-b]indol-3-yl)propionitrile

A 267 mg portion of 3-(2,4-dimethyl-9H-pyrido[2,3-b]indol-3-yl)propanamide synthesized in Reference Example 43 was dissolved in 2.7 ml of DMF, and this was mixed with 0.11 ml of phosphorus oxychloride under ice-cooling and stirred for 1.5 hours. Ethyl acetate and sodium bicarbonate aqueous solution were added to the reaction liquid in that order, and the formed solid was collected by filtration and dried to obtain 92 mg of the title compound as a pale red solid. Also, the filtrate was extracted with ethyl acetate and washed with saturated brine, and then the organic layer was dried with anhydrous magnesium sulfate. By evaporating the solvent under a reduced pressure, 142 mg of the title compound was obtained as a colorless solid.

FAB-MS m/z: 250 (M⁺+1)

### Reference Example 45

### ethyl (+)-3-(9-{4-[(S)-2-(tert-butoxy)-2-oxo-1-(tetrahydro-2H-pyran-4-yl)ethyl]benzyl}-2,4-dimethyl-9H-pyrido[2,3-b]indol-3-yl)propanoate

A 7.7 g portion of ethyl 3-(2,4-dimethyl-9H-pyrido[2,3-b]indol-3-yl)propanoate synthesized in Reference Example 18 was dissolved in 150 ml of N,N-dimethylformamide (DMF), 3.2 g of potassium tert-butoxide was added thereto while cooling to 0° C., and then this was stirred at room temperature for 30 minutes. After cooling the reaction liquid again to 0° C., 30 ml of a DMF solution of 10.0 g of tert-butyl (2S)-(4-bromomethylphenyl)(tetrahydro-2H-pyran-4-yl)acetate synthesized in Reference Example 7 was gradually added dropwise thereto. The reaction liquid was restored to room temperature and stirred at the same temperature for 8 hours. After evaporating the solvent under a reduced pressure, the residue was mixed with 300 ml of chloroform and washed with 300 ml of saturated brine, and then the organic layer was dried with anhydrous magnesium sulfate. The solvent was evaporated under a reduced pressure, and the residue was applied to a silica gel column chromatography and eluted with ethyl acetate/n-hexane (1/5→1/2, v/v) to obtain 8.1 g of the title compound as a pale yellow solid.

In the same manner as in Reference Example 45, compounds shown in the following Table 2 were synthesized.

TABLE 2

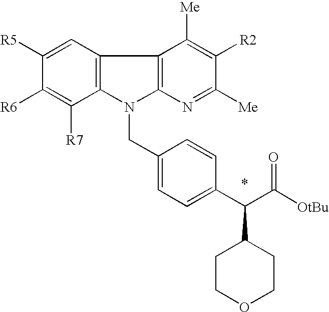

| | Configu- | | | | | |
|---|---|---|---|---|---|---|
| Rex | ration | R2 | R5 | R6 | R7 | DATA |
| 45 | S | (CH2)2CO2Et | H | H | H | FAB-MS m/z:585(M⁺ + 1) |
| | | | | | | [α]25, D = 10.7(c = 1.00, CHCl3) |
| 46 | S | H | H | H | H | FAB-MS m/z:485(M⁺ + 1) |
| 47 | S | H | Cl | CF3 | H | Used in the next reaction without |
| | | | | | | purification. |
| 48 | S | H | H | CO2Me | H | FAB-MS m/z:543(M⁺ + 1) |
| 49 | S | (CH2)3CH3 | H | H | H | FAB-MS m/z:541(M⁺ + 1) |
| 50 | S | (CH2)2CO2Et | F | H | H | FAB-MS m/z:603(M⁺ + 1) |
| 51 | S | (CH2)2CO2Et | H | F | H | FAB-MS m/z:603(M⁺ + 1) |
| 52 | S | (CH2)2CO2Et | H | H | F | FAB-MS m/z:603(M⁺ + 1) |
| 53 | S | (CH2)2CO2Et | Cl | H | H | FAB-MS m/z:619(M⁺ + 1) |
| 54 | S | (CH2)2CO2Et | Me | H | H | FAB-MS m/z:599(M⁺ + 1) |
| 55 | S | (CH2)2CO2H | MeO | H | H | FAB-MS m/z:615(M⁺ + 1) |
| 56 | S | (CH2)2CO2Et | Cl | CF3 | H | FAB-MS m/z:687(M⁺ + 1) |
| 57 | S | (CH2)2CO2Et | H | CN | H | FAB-MS m/z:610(M⁺ + 1) |
| 58 | S | (CH2)2CO2Et | H | SO2NMe2 | H | FAB-MS m/z:692(M⁺ + 1) |
| 59 | S | (CH2)2CO2CH2Ph | H | H | H | FAB-MS m/z:647(M⁺ + 1) |
| | | | | | | [α]25, D = 8.3(c = 0.30, CHCl3) |
| 60 | S | (CH2)2CO2Me | H | CO2Me | H | FAB-MS m/z:629(M⁺ + 1) |
| 61 | S | (CH2)2NMe2 | H | H | H | FAB-MS m/z:556(M⁺ + 1) |
| 62 | S | 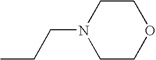 | H | H | H | FAB-MS m/z:598(M⁺ + 1) |
| 63 | S | (CH2)CONH2 | H | H | H | FAB-MS m/z:556(M⁺ + 1) |

### Reference Example 64

### (S)-(+)-(4-{[3-(3-ethoxy-3-oxopropyl)-2,4-dimethyl-9H-pyrido[2,3-b]indol-9-yl-]methyl}phenyl(tetrahydro-2H-pyran-4-yl)acetic acid

A 20.4 g portion of ethyl (+)-3-(9-{4-[(S)-2-(tert-butoxy)-2-oxo-1-(tetrahydro-2H-pyran-4-yl)ethyl]benzyl}-2,4-dimethyl-9H-pyrido[2,3-b]indol-3-yl)propanoate synthesized in Reference Example 45 was dissolved in 100 ml of 1,2-dichloroethane, 100 ml of trifluoroacetic acid was added thereto at room temperature, and this was stirred for 1 hour. After evaporation of the reaction liquid under a reduced pressure, 200 ml of ethyl acetate and saturated sodium bicarbonate aqueous solution were added to the residue. The reaction liquid was neutralized with aqueous citric acid, and then the formed solid was collected by filtration to obtain 5.45 g of the title compound as a colorless solid. Also, the filtrate was extracted with ethyl acetate and washed with saturated brine, and then the organic layer was dried with anhydrous magnesium sulfate. By evaporating the solvent under a reduced pressure and recrystallizing the residue from ethyl acetate, 7.47 g of the title compound was obtained as colorless crystals.

In the same manner as in Reference Example 64, compounds shown in the following Table 3 were synthesized.

TABLE 3

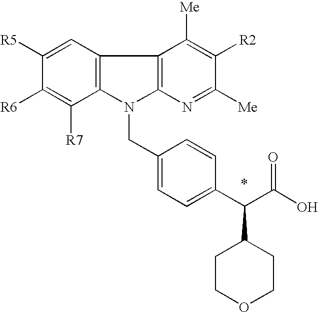

| | Configu- | | | | | |
|---|---|---|---|---|---|---|
| Rex | ration | R2 | R5 | R6 | R7 | DATA |
| 64 | S | (CH2)2CO2Et | H | H | H | FAB-MS m/z:529(M⁺ + 1) |
| | | | | | | [α]25, D = 24.5(c = 1.00, CHCl3) |
| 65 | S | H | H | H | H | FAB-MS m/z:429(M⁺ + 1) |
| 66 | S | H | Cl | CF3 | H | FAB-MS m/z:531(M⁺ + 1) |
| 67 | S | H | H | CO2Me | H | FAB-MS m/z:487(M⁺ + 1) |
| 68 | S | (CH2)3CH3 | H | H | H | FAB-MS m/z:485(M⁺ + 1) |
| 69 | S | (CH2)2CO2Et | F | H | H | FAB-MS m/z:547(M⁺ + 1) |
| 70 | 5 | (CH2)2CO2Et | H | F | H | FAB-MS m/z:547(M⁺ + 1) |
| 71 | S | (CH2)2CO2Et | H | H | F | FAB-MS m/z:547(M⁺ + 1) |
| 72 | S | (CH2)2CO2Et | Cl | H | H | FAB-MS m/z:563(M⁺ + 1) |
| 73 | S | (CH2)2CO2Et | Me | H | H | FAB-MS m/z:543(M⁺ + 1) |
| 74 | 5 | (CH2)2CO2H | MeO | H | H | FAB-MS m/z:559(M⁺ + 1) |
| 75 | S | (CH2)2CO2Et | Cl | CF3 | H | FAB-MS m/z:631(M⁺ + 1) |
| 76 | S | (CH2)2CO2Et | H | CN | H | FAB-MS m/z:554(M⁺ + 1) |
| 77 | S | (CH2)2CO2Et | H | SO2NMe2 | H | FAB-MS m/z:636(M⁺ + 1) |
| 78 | S | (CH2)2CO2CH2Ph | H | H | H | FAB-MS m/z:591(M⁺ + 1) |
| | | | | | | [α]25, D = 20.6(c = 1.00, CHCl3) |
| 79 | S | (CH2)2CO2Me | H | C2O2Me | H | FAB-MS m/z:573(M⁺ + 1) |
| 80 | S | (CH2)2NMe2 | H | H | H | FAB-MS m/z:500(M⁺ + 1) |
| 81 | S | 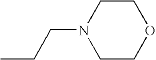 | H | H | H | FAB-MS m/z:542(M⁺ + 1) |
| 82 | S | (CH2)2CONH2 | H | H | H | FAB-MS m/z:500(M⁺ + 1) |

### Reference Example 83

### Methyl (9-{4-[2-(benzyl-tert-butoxycarbonylamino)-2-oxo-1-(tetrahydro-2H-pyran-4-yl)ethyl]benzyl}-2,4-dimethyl-9H-pyrido[2,3-b]indol-3-yl)acetate

A 320 mg portion of methyl 3-(2,4-dimethyl-9H-pyrido[2,3-b]indol-3-yl)acetate synthesized in Reference Example 22 was dissolved in 3 ml of N,N-dimethylformamide (DMF), 134 mg of potassium tert-butoxide was added thereto with cooling to 0° C., and then this was stirred at room temperature for 15 minutes. The reaction liquid was again cooled to 0° C., and then 2 ml of a DMF solution of 600 mg of tert-butyl (±)-N-benzyl-[(4-bromomethylphenyl)-2-(tetrahydro-2H-pyran-4-yl)]ethanoylcarbamate synthesized in Reference Example 10 was gradually added dropwise thereto. The reaction liquid was restored to room temperature and stirred overnight at the same temperature. The reaction liquid was mixed with ethyl acetate and distilled water and extracted with ethyl acetate. The organic layer was washed with distilled water and saturated brine and dried with anhydrous magnesium sulfate, and then the solvent was evaporated under a reduced pressure. The residue was applied to a silica gel column chromatography and eluted with a mixed solvent of hexane/ethyl acetate (9/1→4/1, v/v) to obtain 503 mg of the title compound as a colorless oil.

FAB-MS m/z: 690 (M⁺+1)

In the same manner as in Reference Example 83, compounds shown in the following Table 4 were synthesized.

TABLE 4

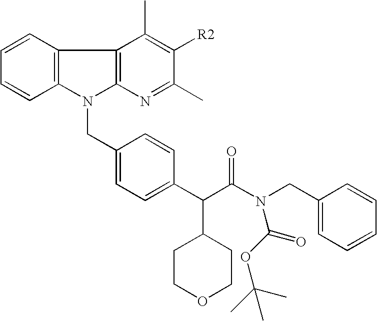

| Rex | Configuration | R2 | DATA |
|---|---|---|---|
| 84 | ± | (CH2)3CO2Et | FAB-MS m/z:732(M⁺ + 1) |
| 85 | ± | (CH2)2CN | FAB-MS m/z:671(M⁺ + 1) |

### Reference Example 86

### (4-Methylphenyl)(tetrahydro-2H-pyran-4-yl)acetic acid

(1) Pyridine (7.6 mole equiv.), p-toluenesulfonyl chloride (2.2 mole equiv.) and water (7.6 mole equiv.) were added to 5 g of tetrahydropyran-4-ol and stirred overnight at 24° C. This reaction liquid was mixed with 60 ml of water, extracted with 60 ml of toluene and washed with 60 ml of 2 M hydrochloric acid and 40 ml of water in that order, and then the organic layer was concentrated to dryness to obtain 8.98 g of tetrahydro-2H-pyran-4-yl 4-methylbenzenesulfonate.

¹H NMR: δ=7.83-7.81 (2H, d), 7.49-7.47 (2H, d), 4.75-4.65 (1H, m), 3.74-3.36 (4H, m), 2.42 (s, 3H), 1.78-1.52 (4H, m)

(2) To 10 ml of THF were added 17.7 ml of 1.58 M n-BuLi n-hexane solution and 3.1 g of diisopropylamine at 0° C. or lower (LDA preparation), and then 2 g of p-tolylacetic acid (by dissolving in 8 ml of THF) was added thereto and stirred for 1 hour. Subsequently, 3.41 g of tetrahydro-2H-pyran-4-yl 4-methylbenzenesulfonate was added thereto at 0° C. or lower, and this was stirred at 24° C. for 6 hours. This reaction liquid was mixed with 10 ml of water, washed twice with 12 ml of ethyl acetate, adjusted to pH 4 by adding concentrated hydrochloric acid and then extracted twice with 12 ml of ethyl acetate, the extract was concentrated, and then the residue was dissolved by adding 7 ml of ethanol and crystallized by adding 14 ml of water to obtain 2.65 g of (4-methylphenyl)(tetrahydro-2H-pyran-4-yl)acetic acid.

¹H NMR: δ=12.32 (1H, s), 7.20-7.12 (4H, q), 3.86-3.14 (5H, m), 2.27 (3H, s), 2.08 (1H, m), 1.69-1.00 (4H, m)

### Reference Example 87

### 2-Imino-2,3-dihydro-1H-indole hydrochloride

(1) In accordance with a literature (Grob, C. A.; Helv Chim Acta 1961, 44, 1748), (2-nitrophenyl)acetonitrile was synthesized from 1-chloro-2-nitrobenzene via ethyl (±)-cyano(2-nitrophenyl)acetate.

Subsequently, catalytic reduction of 3.37 g (20.78 mmol) of (2-nitrophenyl)acetonitrile was carried out in 17 ml of methanol using 0.15 g of 10% Pd—C (54% wet). The catalyst was removed by filtration, the solvent was evaporated, 20 ml of diisopropyl ether was added to the thus obtained residue, and then the crystals were collected by filtration to obtain 2.05 g of (2-aminophenyl)acetonitrile.

FAB-MS (pos.): m/z 133

(2) A 1.34 g portion of (2-aminophenyl)acetonitrile was dissolved in 10 ml of THF, and this was mixed with concentrated hydrochloric acid and heated under reflux for 2 hours. The solvent was evaporated, the thus obtained residue was suspended by adding 5 ml of ethanol and 15 ml of diisopropyl ether thereto, and then the crystals were collected by filtration to obtain 1.42 g of 2-imino-2,3-dihydro-1H-indole hydrochloride.

¹H NMR: δ=12.46 (1H, s), 10.25 (1H, s), 10.00 (1H, s); 7.42-7.11 (4H, m), 4.18 (2H, s)

### Example 1

### Ethyl (−)-3-(2,4-dimethyl-9-{4-[(S)-2-oxo-2-(4-pyridin-2-ylpiperazin-1-yl)-1-(tetrahydro-2H-pyran-4-yl)ethyl]benzyl}-9H-pyrido[2,3-b]indol-3-yl)propanoate

A 2.0 g portion of (S)-(+)-(4-{[3-(3-ethoxy-3-oxopropyl)-2,4-dimethyl-9H-pyrido[2,3-b]indol-9-yl]methyl}phenyl)(tetrahydro-2H-pyran-4-yl)acetic acid synthesized in Reference Example 64 was dissolved in 30 ml of DMF, to which were added 560 mg of 1-hydroxybenzotriazole (HOBt), 780 mg of 1-ethyl-3-(3-dimethylaminopropyl)carbodiimide hydrochloride (WSC HCl), 680 mg of 1-(2-pyridyl)piperazine and 580 mg of triethylamine in that order at room temperature, and this was stirred overnight at the same temperature. The solvent was evaporated under a reduced pressure, the residue was mixed with 100 ml of ethyl acetate and washed with water, saturated sodium bicarbonate aqueous solution and saturated brine, and then the organic layer was dried with anhydrous magnesium sulfate and the solvent was evaporated under a reduced pressure. The residue was applied to a silica gel column chromatography and eluted with a mixed solvent of chloroform/methanol (100/1, v/v) to obtain 2.42 g of the title compound as a pale yellow foam substance. By crystallizing this from ethanol, 1.85 g of the title compound was obtained as colorless crystals.

### Example 2

### Benzyl (+)-3-(2,4-dimethyl-9-{4-[(S)-2-oxo-2-(4-propylpiperazin-1-yl)-1-(tetrahydro-2H-pyran-4-yl)ethyl]benzyl}-9H-pyrido[2,3-b]indol-3-yl)propanoate

A 1.8 g portion of (S)-(+)-(4-{[3-(3-benzyloxy-3-oxopropyl)-2,4-dimethyl-9H-pyrido[2,3-b]indol-9-yl]methyl}phenyl)(tetrahydro-2H-pyran-4-yl)acetic acid synthesized in Reference Example 78 was dissolved in 40 ml of DMF, to which were added 460 mg of HOBt, 650 mg of WSC HCl, 980 mg of 1-(n-propyl)piperazine dihydrobromide and 930 mg of triethylamine in that order at room temperature, and this was stirred at the same temperature for 10 hours. The solvent was evaporated under a reduced pressure, the residue was mixed with 150 ml of ethyl acetate and washed with water, saturated sodium bicarbonate aqueous solution and saturated brine, and then the organic layer was dried with anhydrous magnesium sulfate and the solvent was evaporated under a reduced pressure. The residue was applied to a silica gel column chromatography and eluted with a mixed solvent of chloroform/methanol (20/1, v/v) to obtain 1.85 g of the title compound as a colorless foam substance. By crystallizing a 300 mg portion of this from ethanol, 148 mg of the title compound was obtained as colorless crystals.

In the same manner as in Example 1, the compounds shown in Table 5 were synthesized.

TABLE 5

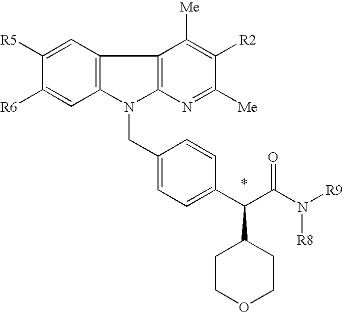

| | Configu- | | | | | |
|---|---|---|---|---|---|---|
| Ex. | ration | R2 | R5 | R6 | NR8R9 | DATA |
| 1 | S | (CH2)2CO2Et | H | H | 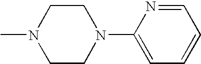 | FAB-MS m/z:674(M⁺ + 1) [α]25, D = 25.6(c = 0.50, CHCl3) |
| 2 | S | (CH2)2CO2CH2Ph | H | H | 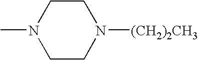 | FAB-MS m/z:701(M⁺ + 1) [α]25, D = 9.5(c = 0.40, CHCl3) |
| 3 | S | H | H | H | NHCH2Ph | FAB-MS m/z:518(M⁺ + 1) |
| 4 | S | H | Cl | CF3 | NHCH2Ph | FAB-MS m/z:620(M⁺ + 1) |
| 5 | S | H | H | CO2Me | NHCH2Ph | FAB-MS m/z:576(M⁺ + 1) |
| 6 | S | (CH2)2CO2CH2Ph | H | H | NHNHPh | FAB-MS m/z:681(M⁺ + 1) |
| 7 | S | (CH2)3CH3 | H | H | NHCH2Ph | FAB-MS m/z:574(M⁺ + 1) |
| 8 | S | (CH2)2CO2Et | H | H | NHCH2Ph | FAB-MS m/z:618(M⁺ + 1) |
| 9 | S | (CH2)2CO2CH2Ph | H | H | NHCH2Ph | FAB-MS m/z:680(M⁺ + 1) |
| | | | | | | [α]25, D = 63.0(c = 1.00, DMF) |
| 10 | S | (CH2)2CO2Et | Cl | CF3 | NHCH2Ph | FAB-MS m/z:720(M⁺ + 1) |
| 11 | S | (CH2)2CO2Et | H | H | 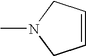 | FAB-MS m/z:580(M⁺ + 1) |
| 12 | S | (CH2)2CO2Et | H | H | 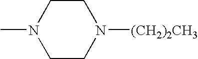 | FAB-MS m/z:639(M⁺ + 1) [α]25, D = 10.0(c = 0.50, CHCl3) |
| 13 | S | (CH2)2CO2Et | H | H | 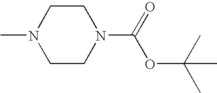 | FAB-MS m/z:697(M⁺ + 1) |
| 14 | S | (CH2)2NMe2 | H | H | NHCH2Ph | FAB-MS m/z:589(M⁺ + 1) |
| 15 | S | (CH2)2CONH2 | H | H | NHCH2Ph | FAB-MS m/z:589(M⁺ + 1) |

### Example 16

### Ethyl (+)-3-(2,4-dimethyl-9-{4-[(S)-2-oxo-2-(piperazin-1-yl)-1-(tetrahydro-2H-pyran-4-yl)ethyl]benzyl}-9H-pyrido[2,3-b]indol-3-yl)propanoate dihydrochloride

A 2.11 g portion of 4-(tert-butoxycarbonyl)piperazine was dissolved in 40 ml of DMF, to which were added 4.0 g of (S)-(+)-(4-{[3-(3-ethoxy-3-oxopropyl)-2,4-dimethyl-9H-pyrido[2,3-b]indol-9-yl]methyl}phenyl)(tetrahydro-2H-pyran-4-yl)acetic acid synthesized in Reference Example 64, 1.74 g of 1-ethyl-3-(3-dimethylaminopropyl)carbodiimide hydrochloride (WSC HCl) and 1.23 g of 1-hydroxybenzotriazole (HOBt) in that order at room temperature, and this was stirred overnight at the same temperature. The reaction liquid was mixed with ethyl acetate and sodium bicarbonate aqueous solution and extracted with ethyl acetate. The organic layer was washed with water, 1.0 M hydrochloric acid and saturated brine and dried with anhydrous magnesium sulfate, and then the solvent was evaporated under a reduced pressure to obtain a colorless foam substance. The obtained foam substance was dissolved in 20 ml of ethyl acetate, 20 ml of an ethyl acetate solution of 4 M hydrogen chloride was added thereto at room temperature and stirred for 1 hour. The reaction liquid was evaporated under a reduced pressure and then washed with ethyl acetate to obtain 5.07 g of the title compound as a colorless solid.

FAB-MS m/z: 597 (M⁺+1)

### Example 17

### (−)-3-(2,4-Dimethyl-9-{4-[(S)-2-oxo-2-(4-pyridin-2-ylpiperazin-1-yl)-1-(tetrahydro-2H-pyran-4-yl)ethyl]benzyl}-9H-pyrido[2,3-b]indol-3-yl)propanoic acid

To 1.84 g of ethyl (−)-3-(2,4-dimethyl-9-{4-[(S)-2-oxo-2-(4-pyridin-2-ylpiperazin-1-yl)-1-(tetrahydro-2H-pyran-4-yl)ethyl]benzyl}-9H-pyrido[2,3-b]indol-3-yl)propanoate synthesized in Example 1 were added 100 ml of methanol and 15 ml of water, subsequently adding thereto 830 mg of anhydrous potassium carbonate, and this was stirred at 70° C. for 3 hours. After ice-cooling, 5.95 ml of 1 M hydrochloric acid was added thereto, and the solvent was evaporated under a reduced pressure. The residue was mixed with chloroform and washed with saturated brine, and then the organic layer was dried with anhydrous magnesium sulfate and the solvent was evaporated under a reduced pressure. The residue was applied to a silica gel column chromatography and eluted with a mixed solvent of chloroform/methanol (20/1, v/v) to obtain 1.8 g of the title compound as a colorless foam substance. By crystallizing this from ethanol and then from acetone, 710 mg of the title compound was obtained as colorless crystals.

### Example 18

### (+)-3-(2,4-Dimethyl-9-{4-[(S)-2-oxo-2-(4-propylpiperazin-1-yl)-1-(tetrahydro-2H-pyran-4-yl)ethyl]benzyl}-9H-pyrido[2,3-b]indol-3-yl)propanoic acid

A 1.96 g portion of benzyl (+)-3-(2,4-dimethyl-9-{4-[(S)-2-oxo-2-(4-propylpiperazin-1-yl)-1-(tetrahydro-2H-pyran-4-yl)ethyl]benzyl}-9H-pyrido[2,3-b]indol-3-yl)propanoate synthesized in Example 2 was dissolved in 50 ml of tetrahydrofuran, and 300 mg of 10% palladium-carbon was added thereto under a stream of argon. Hydrogen gas was introduced into the reaction system, and stirred overnight at room temperature. The reaction system was purged with argon gas, the insoluble matter was removed by filtration using Celite, and then the solvent was evaporate under a reduced pressure. The residue was applied to a silica gel column chromatography and eluted with a mixed solvent of chloroform/methanol (20/1, v/v) to obtain 1.75 g of the title compound as a colorless foam substance. By crystallizing this from ethyl acetate and ethanol in that order, 765 mg of the title compound was obtained as colorless crystals.

### Example 19

### (−)-3-(9-{4-[(S)-(benzylcarbamoyl)(tetrahydro-2H-pyran-4-yl)methyl]benzyl}-2,4-dimethyl-9H-pyrido[2,3-b]indol-3-yl)propanoic acid

A 3.2 g portion of benzyl (−)-3-(9-{4-[(S)-(benzylcarbamoyl)(tetrahydro-2H-pyran-4-yl)methyl]benzyl}-2,4-dimethyl-9H-pyrido[2,3-b]indol-3-yl)propanoate synthesized in Example 9 was dissolved in 120 ml of tetrahydrofuran, and 500 mg of 10% palladium-carbon was added thereto under a stream of argon. Hydrogen gas was introduced into the reaction system, and stirred at room temperature for 2 days. The reaction system was purged with argon gas, the insoluble matter was removed by filtration using Celite, and then the solvent was evaporate under a reduced pressure. The residue was applied to a silica gel column chromatography and eluted with a mixed solvent of chloroform/methanol (20/1, v/v) to obtain 3.31 g of the title compound as a colorless foam substance. By crystallizing this from ethanol, 2.58 g of the title compound was obtained as colorless crystals.

### Example 20

### (+)-3-(9-{4-[(S)-2-(4-cyclopropylmethylpiperazin-1-yl)-2-oxo-1-(tetrahydro-2H-pyran-4-yl)ethyl]benzyl-2,4-dimethyl-9H-pyrido[2,3-b]indol-3-yl}propanoic acid

A 94 mg portion of cyclopropylaldehyde was dissolved in 6 ml of 1,2-dichloroethane, and 300 mg of ethyl (+)-3-(2,4-dimethyl-9-{4-[(S)-2-oxo-2-(piperazin-1-yl)-1-(tetrahydro-2H-pyran-4-yl)ethyl]benzyl}-9H-pyrido[2,3-b]indol-3-yl)propanoate dihydrochloride synthesized in Example 16 was added thereto and stirred for 1 hour. The reaction liquid was mixed with 285 mg of sodium triacetoxyborohydride at the same temperature and stirred for 1 hour. The reaction liquid was mixed with chloroform and saturated sodium bicarbonate aqueous solution and then extracted with chloroform. The organic layer was washed with saturated brine and dried with anhydrous magnesium sulfate, and then the solvent was evaporated under a reduced pressure. The residue was applied to a silica gel column chromatography and eluted with a mixed solvent of chloroform/methanol (99/1, v/v) to obtain 261 mg of a colorless foam substance. The obtained foam substance was dissolved in 12 ml of ethanol and 6 ml of distilled water, 196 mg of potassium carbonate was added thereto at room temperature. The reaction liquid was stirred overnight at 80° C., and then evaporated under a reduced pressure. The residue was mixed with 2.8 ml of 1 M hydrochloric acid and extracted with chloroform. The organic layer was dried with anhydrous magnesium sulfate, the solvent was evaporated under a reduced pressure, and then the residue was recrystallized from a mixed solvent of ethyl acetate and n-hexane, thereby obtaining 194 mg of the title compound as colorless crystals.

In the same manner as in Examples 17, 18, 19 or 20, the compounds shown in Table 6 below were synthesized.

TABLE 6

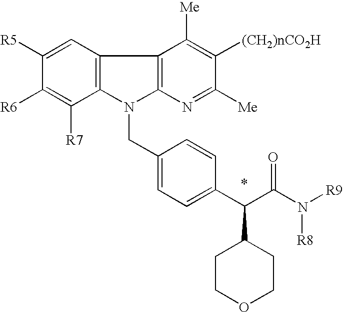

| | Configu- | | | | | | |
|---|---|---|---|---|---|---|---|
| Ex | ration | n | R5 | R6 | R7 | NR8R9 | DATA |
| 17 | S | 2 | H | H | H | 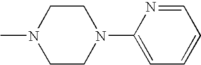 | FAB-MS m/z:646(M⁺ + 1) [α]25, D = 23.9(c = 1.00, CHCl3) |
| 18 | S | 2 | H | H | H | 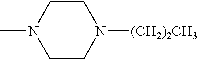 | FAB-MS m/z:611(M⁺ + 1) [α]25, D = 18.0(c = 1.00, CHCl3) |
| 19 | S | 2 | H | H | H | NHCH2Ph | FAB-MS m/z:590(M⁺ + 1) |
| | | | | | | | [α]25, D = 7.2(c = 0.50, DMF) |
| 20 | S | 2 | H | H | H | 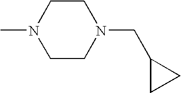 | FAB-MS m/z:623(M⁺ + 1) [α]25, D = 16.3(c = 1.00, CHCl3) |
| 21 | S | 2 | H | H | H | NHNHPh | FAB-MS m/z:591(M⁺ + 1) |
| 22 | S | 3 | H | H | H | NHCH2Ph | FAB-MS m/z:604(M⁺ + 1) |
| 23 | S | 2 | F | H | H | NHCH2Ph | FAB-MS m/z:608(M⁺ + 1) |
| 24 | S | 2 | H | F | H | NHCH2Ph | FAB-MS m/z:608(M⁺ + 1) |
| 25 | S | 2 | H | H | F | NHCH2Ph | FAB-MS m/z:608(M⁺ + 1) |
| 26 | S | 2 | Cl | H | H | NHCH2Ph | FAB-MS m/z:624(M⁺ + 1) |
| 27 | S | 2 | Cl | CF3 | H | NHCH2Ph | FAB-MS m/z:692(M⁺ + 1) |
| 28 | S | 2 | Me | H | H | NHCH2Ph | FAB-MS m/z:604(M⁺ + 1) |
| 29 | S | 2 | MeO | H | H | NHCH2Ph | FAB-MS m/z:620(M⁺ + 1) |
| 30 | S | 2 | H | CN | H | NHCH2Ph | FAB-MS m/z:615(M⁺ + 1) |
| 31 | S | 2 | H | CO2Me | H | NHCH2Ph | FAB-MS m/z:648(M⁺ + 1) |
| 32 | S | 2 | H | H | H | NHCH2(2-F-Ph) | FAB-MS m/z:608(M⁺ + 1) |
| 33 | S | 2 | H | H | H | NHCH2(2-Cl-Ph) | FAB-MS m/z:625(M⁺ + 1) |
| 34 | S | 2 | H | H | H | NHCH2(2-MeO-Ph) | FAB-MS m/z:620(M⁺ + 1) |
| 35 | S | 2 | H | H | H | 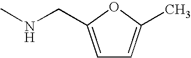 | FAB-MS m/z:594(M⁺ + 1) |
| 36 | S | 2 | H | H | H | 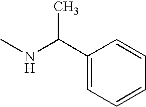 | FAB-MS m/z:604(M⁺ + 1) |
| 37 | S | 2 | H | H | H | N(CH3)2 | FAB-MS m/z:528(M⁺ + 1) |
| 38 | S | 2 | H | H | H | 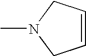 | FAB-MS m/z:552(M⁺ + 1) |
| 39 | S | 2 | H | H | H | 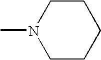 | FAB-MS m/z:568(M⁺ + 1) |
| 40 | S | 2 | H | H | H | 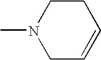 | FAB-MS m/z:564(M⁺ + 1) |
| 41 | S | 2 | H | H | H | 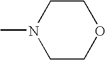 | FAB-MS m/z:570(M⁺ + 1) |
| 42 | S | 2 | H | H | H | 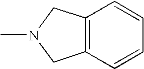 | FAB-MS m/z:602(M⁺ + 1) |
| 43 | S | 2 | H | H | H | 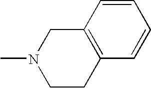 | FAB-MS m/z:616(M⁺ + 1) |
| 44 | S | 2 | H | H | H | 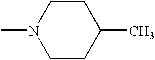 | FAB-MS m/z:582(M⁺ + 1) |
| 45 | S | 2 | H | H | H | 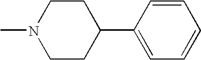 | FAB-MS m/z:644(M⁺ + 1) |
| 46 | S | 2 | H | H | H | 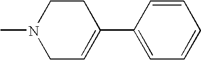 | FAB-MS m/z:642(M⁺ + 1) |
| 47 | S | 2 | H | H | H | 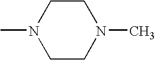 | FAB-MS m/z:583(M⁺ + 1) |
| 48 | S | 2 | H | H | H | 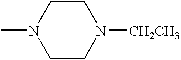 | FAB-MS m/z:597(M⁺ + 1) |
| 49 | S | 2 | H | H | H | 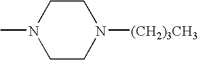 | FAB-MS m/z:625(M⁺ + 1) |
| 50 | S | 2 | H | H | H | 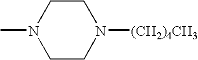 | FAB-MS m/z:639(M⁺ + 1) |
| 51 | S | 2 | H | H | H | 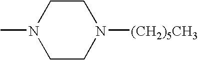 | FAB-MS m/z:653(M⁺ + 1) |
| 52 | S | 2 | H | H | H | 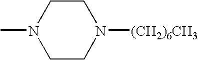 | FAB-MS m/z:667(M⁺ + 1) |
| 53 | S | 2 | H | H | H | 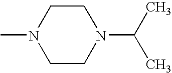 | FAB-MS m/z:611(M⁺ + 1) |
| 54 | S | 2 | H | H | H | 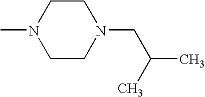 | FAB-MS m/z:625(M⁺ + 1) |
| 55 | S | 2 | H | H | H | 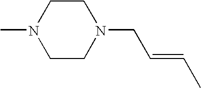 | FAB-MS m/z:623(M⁺ + 1) |
| 56 | S | 2 | H | H | H | 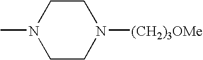 | FAB-MS m/z:641(M⁺ + 1) |
| 57 | S | 2 | H | H | H | 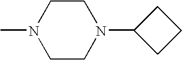 | FAB-MS m/z:623(M⁺ + 1) |
| 58 | S | 2 | H | H | H | 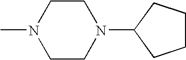 | FAB-MS m/z:637(M⁺ + 1) |
| 59 | S | 2 | H | H | H | 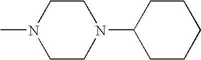 | FAB-MS m/z:651(M⁺ + 1) |
| 60 | S | 2 | H | H | H | 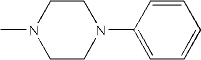 | FAB-MS m/z:645(M⁺ + 1) |
| 61 | S | 2 | H | H | H | 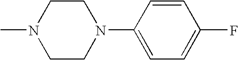 | FAB-MS m/z:663(M⁺ + 1) |
| 62 | S | 2 | H | H | H | 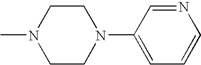 | FAB-MS m/z:646(M⁺ + 1) |
| 63 | S | 2 | H | H | H | 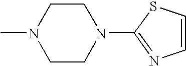 | FAB-MS m/z:652(M⁺ + 1) |
| 64 | S | 2 | H | H | H | 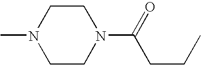 | FAB-MS m/z:639(M⁺ + 1) |
| 65 | S | 2 | H | H | H | 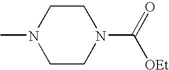 | FAB-MS m/z:641(M⁺ + 1) |
| 66 | S | 2 | H | H | H | 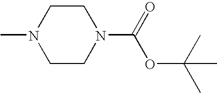 | FAB-MS m/z:669(M⁺ + 1) |
| 67 | S | 2 | H | H | H | 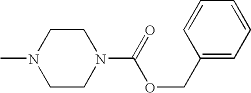 | FAB-MS m/z:703(M⁺ + 1) |
| 68 | S | 2 | H | H | H | 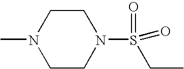 | FAB-MS m/z:661(M⁺ + 1) |

### Example 69

### (S)-(−)-N-benzyl-2-(4-{[2,4-dimethyl-3-(3-hydroxypropyl)-9H-pyrido[2,3-b]indol-9-yl]methyl}phenyl)-2-(tetrahydro-2H-pyran-4-yl)acetamide

A 295 mg portion of (−)-3-(9-{4-[(S)-(benzylcarbamoyl)(tetrahydro-2H-pyran-4-yl)methyl]benzyl}-2,4-dimethyl-9H-pyrido[2,3-b]indol-3-yl)propanoic acid synthesized in Example 19 was dissolved in 10 ml of tetrahydrofuran, to which was added 3.0 ml of a tetrahydrofuran solution of 1.0 M borane-tetrahydrofuran complex at 0° C. in a stream of argon. After 3.5 hours of stirring at room temperature, 15 ml of water and 6.0 g of potassium carbonate were added thereto and stirred at room temperature for 1 hour. The reaction liquid was extracted with ethyl acetate, washed with saturated brine and then dried with anhydrous magnesium sulfate. The solvent was evaporate under a reduced pressure, and the residue was applied to a silica gel column chromatography and eluted with chloroform/methanol (50/1, v/v) to obtain 296 mg of the title compound as a colorless solid. By crystallizing this from ethanol, 217 mg of the title compound was obtained as crystals.

FAB-MS m/z: 576 (M⁺+1), [α]25.0. D=−72.0 (c=1.00, DMF)

### Example 70

### (±)-N-benzyl-2-(4-{[3-(2-cyanoethyl)-2,4-dimethyl-9H-pyrido[2,3-b]indol-9-yl]methyl}phenyl)-2-(tetrahydro-2H-pyran-4-yl)acetamide

A 1.1 g portion of tert-butyl (±)-N-benzyl-[2-(4-{[3-(2-cyanoethyl)-2,4-dimethyl-9H-pyrido[2,3-b]indol-9-yl]methyl}phenyl)-2-(tetrahydro-2H-pyran-4-yl)ethanoyl]carbamate synthesized in Reference Example 85 was dissolved in 10 ml of 1,2-dichloroethane, and 5 ml of trifluoroacetic acid was added thereto at room temperature and stirred for 3 hours. After evaporation of the reaction liquid under a reduced pressure, the residue was mixed with ethyl acetate and sodium bicarbonate aqueous solution and extracted with ethyl acetate. The organic layer was washed with distilled water and saturated brine and dried with anhydrous magnesium sulfate, and then the solvent was evaporated under a reduced pressure. By recrystallizing the residue from ethyl acetate, 728 mg of the title compound was obtained as colorless crystals.

FAB-MS m/z: 571 (M⁺+1)

### Example 71

### Sodium (±)-(9-{4-[benzylcarbamoyl-(tetrahydro-2H-pyran-4-yl)methyl]benzyl}-2,4-dimethyl-9H-pyrido[2,3-b]indol-3-yl)acetate

A 500 mg portion of methyl (±)-(9-{4-[2-(benzyl-tert-butoxycarbonylamino)-2-oxo-1-(tetrahydro-2H-pyran-4-yl)ethyl]benzyl}-2,4-dimethyl-9H-pyrido[2,3-b]indol-3-yl)acetate synthesized in Reference Example 83 was dissolved in 5 ml of ethyl acetate, and 5 ml of an ethyl acetate solution of 4 M hydrogen chloride was added thereto at room temperature and stirred for 2 hours. After evaporation of the reaction liquid under a reduced pressure, the residue was dissolved in 5 ml of methanol, mixed with 3.6 ml of 1 M sodium hydroxide aqueous solution at room temperature and stirred overnight at room temperature. After evaporation of the reaction liquid under a reduced pressure, ethyl acetate and citric acid aqueous solution were added to the residue, and the formed colorless solid was collected by filtration. The filtrate was extracted with ethyl acetate, the organic layer was washed with distilled water and saturated brine and dried with anhydrous magnesium sulfate, and then the solvent was evaporated under a reduced pressure. The residue was mixed with the colorless solid collected by filtration previously, and washed with ethyl acetate to obtain 240 mg of a colorless solid. The thus obtained 240 mg of colorless solid was dissolved in 5 ml of ethanol, 0.41 ml of 1 M sodium hydroxide aqueous solution was added thereto at room temperature, and the formed solid was collected by filtration and dried to obtain 185 mg of the title compound as a colorless solid.

FAB-MS m/z: 598 (M⁺+1)

In the same manner as in Example 71, the compound of the following Example 72 was synthesized.

### Example 72

### Sodium (±)-(9-{4-[benzylcarbamoyl-(tetrahydro-2H-pyran-4-yl)methyl]benzyl}-2,4-dimethyl-9H-pyrido[2,3-b]indol-3-yl)butanoate

### Example 73

### (±)-N-benzyl-2-[4-({2,4-dimethyl-3-[2-(1H-tetrazol-5-yl)ethyl]-9H-pyrido[2,3-b]indol-9-yl}methyl)phenyl]-2-(tetrahydro-2H-pyran-4-yl)acetamide

A 1.45 g portion of tributyltin azide was dissolved in 5 ml of toluene, 500 mg of (±)-N-benzyl-2-(4-{[3-(2-cyanoethyl)-2,4-dimethyl-9H-pyrido[2,3-b]indol-9-yl]methyl}phenyl)-2-(tetrahydro-2H-pyran-4-yl)acetamide synthesized in Example 70 was added thereto at room temperature, and this was stirred overnight with heating under reflux. To the reaction liquid were added 10 ml of 1 M hydrochloric acid and 20 ml of ethyl acetate. By adding saturated sodium bicarbonate aqueous solution and aqueous citric acid thereto, the formed solid was collected by filtration. After extraction of the filtrate with ethyl acetate, the organic layer was washed with saturated brine and dried with anhydrous magnesium sulfate, and then the solvent was evaporated under a reduced pressure. The residue was mixed with the solid previously collected by filtration and washed with hot hexane. By recrystallizing the obtained colorless solid from ethanol, 272 mg of the title compound was obtained as colorless crystals.

FAB-MS m/z: 614 (M⁺+1)

The compounds shown in the following Tables 7 and 8 were synthesized in the same manner as in the aforementioned Reference Examples and Examples, or as occasion demands, by further employing any operation generally used by those skilled in the art.

TABLE 7

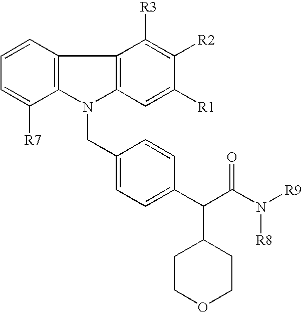

| | Con- | | | | | | |
|---|---|---|---|---|---|---|---|
| | figu- | | | | | | |
| | ra- | | | | | | |
| Ex. | tion | R1 | R2 | R3 | R7 | NR8R9 | DATA |
| 74 | S | H | H | H | H | NHCH2Ph | FAB-MS m/z:489(M⁺ + 1) |
| 75 | S | H | H | H | H | NHCH2(2-Py) | FAB-MS m/z:490(M⁺ + 1) |
| 76 | ± | H | Cl | H | CH2CO2H | NHCH2Ph | FAB-MS m/z:581(M⁺ + 1) |
| 77 | S | Me | H | Me | H | NHCH2Ph | FAB-MS m/z:517(M⁺ + 1) |
| 78 | S | Me | CH2CO2Et | Me | H | NHCH2Ph | FAB-MS m/z:575(M⁺ + 1) |
| 79 | S | Me | CH2CO2H | Me | H | NHCH2Ph | FAB-MS m/z:547(M⁺ + 1) |
| 80 | ± | H | (CH2)2CO2H | H | H | NHCH2Ph | FAB-MS m/z:559(M⁺ + 1) |

TABLE 8

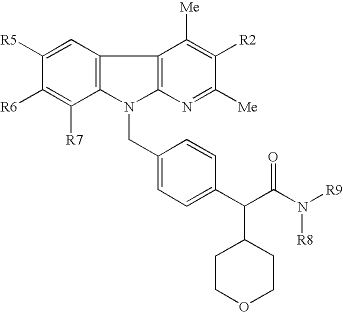

| | Con | | | | | | |
|---|---|---|---|---|---|---|---|
| | figu- | | | | | | |
| | ra- | | | | | | |
| Ex. | tion | R2 | R5 | R6 | R7 | NR8R9 | DATA |
| 81 | S | H | H | H | H | 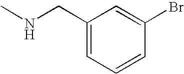 | FAB-MS m/z:596(M⁺ + 1) |
| 82 | S | H | H | H | H | 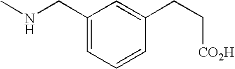 | FAB-MS m/z:590(M⁺ + 1) |
| 83 | S | H | H | H | H | 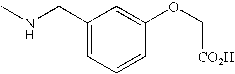 | FAB-MS m/z:564(M⁺ + 1) |
| 84 | S | H | H | H | H | 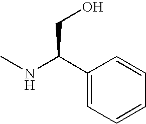 | FAB-MS m/z:548(M⁺ + 1) |
| 85 | S | H | H | CO2H | H | NHCH2Ph | FAB-MS m/z:562(M⁺ + 1) |
| 86 | S | H | H | 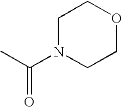 | H | NHCH2Ph | FAB-MS m/z:635(M⁺ + 1) |
| 87 | S | H | H | 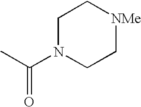 | H | NHCH2Ph | FAB-MS m/z:644(M⁺ + 1) |
| 88 | S | H | Cl | Cl | H | 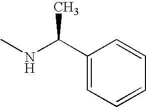 | Column Separation of diastereomers FAB-MS m/z:600(M⁺ + 1) |
| 89 | ± | H | H | H | H | NHNHPh | FAB-MS m/z:519(M⁺ + 1) |
| 93 | ± | H | Cl | Cl | H | NHNHPh | FAB-MS m/z:587(M⁺ + 1) |
| 91 | ± | H | Cl | Cl | H | NHN(Me)Ph | FAB-MS m/z:560(M⁺ + 1) |
| 92 | ± | H | Cl | Cl | H | NHNH(2-Py) | FAB-MS m/z:547(M⁺ + 1) |
| 93 | ± | H | Cl | Cl | H | NHN(Me)(2-Py) | FAB-MS m/z:561(M⁺ + 1) |
| 94 | ± | H | Cl | CF3 | H | NHNHPh | FAB-MS m/z:621(M⁺ + 1) |
| 95 | ± | H | Cl | CF3 | H | NHN(Me)Ph | FAB-MS m/z:635(M⁺ + 1) |
| 96 | ± | H | Cl | CF3 | H | NHNH(2-Py) | FAB-MS m/z:622(M⁺ + 1) |
| 97 | ± | H | Cl | CF3 | H | NHN(Me)(2-Py) | FAB-MS m/z:636(M⁺ + 1) |
| 98 | S | H | H | H | H | N(CH3)2 | FAB-MS m/z:456(M⁺ + 1) |
| 99 | S | H | H | H | H | 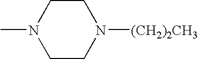 | FAB-MS m/z:539(M⁺ + 1) |
| 100 | S | H | H | H | H | 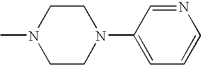 | FAB-MS m/z:574(M⁺ + 1) |
| 101 | ± | CO2Et | H | H | H | NHCH2Ph | FAB-MS m/z:590(M⁺ + 1) |
| 102 | ± | CO2H | H | H | H | NHCH2Ph | FAB-MS m/z:562(M⁺ + 1) |
| 103 | ± | CH2OH | H | H | H | NHCH2Ph | FAB-MS m/z:548(M⁺ + 1) |
| 104 | S | 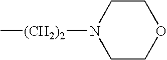 | H | H | H | NHCH2Ph | FAB-MS m/z:263(M⁺ + 1) |
| 105 | ± | (CH2)2CONHMe | H | H | H | NHCH2Ph | FAB-MS m/z:603(M⁺ + 1) |
| 106 | ± | (CH2)2CONMe2 | H | H | H | NHCH2Ph | FAB-MS m/z:617(M⁺ + 1) |
| 107 | ± | (CH2)2CO2Et | F | H | H | NHCH2Ph | FAB-MS m/z:626(M⁺ + 1) |
| 108 | ± | (CH2)2CO2Et | F | H | F | NHCH2Ph | FAB-MS m/z:624(M⁺ + 1) |
| 109 | ± | (CH2)2CO2Et | Me | H | H | NHCH2Ph | FAB-MS m/z:604(M⁺ + 1) |
| 110 | S | (CH2)2CO2H | H | H | H | NHCH3 | FAB-MS m/z:514(M⁺ + 1) |
| 111 | ± | (CH2)2CO2H | F | H | F | NHCH2Ph | FAB-MS m/z:626(M⁺ + 1) |
| 112 | S | (CH2)2CO2H | H | Cl | H | NHCH2Ph | FAB-MS m/z:624(M⁺ + 1) |
| 113 | S | (CH2)2CO2H | Me | H | H | NHCH2Ph | FAB-MS m/z:604(M⁺ + 1) |
| 114 | S | (CH2)2CO2H | H | Me | H | NHCH2Ph | FAB-MS m/z:604(M⁺ + 1) |
| 115 | S | (CH2)2CO2H | H | CF3 | H | NHCH2Ph | FAB-MS m/z:658(M⁺ + 1) |
| 116 | S | (CH2)2CO2H | H | SO2NMe2 | H | NHCH2Ph | FAB-MS m/z:697(M⁺ + 1) |
| 117 | S | (CH2)2CO2H | H | H | H | NHCH2(3-F-Ph) | FAB-MS m/z:608(M⁺ + 1) |
| 118 | S | (CH2)2CO2H | H | H | H | NHCH2(4-F-Ph) | FAB-MS m/z:608(M⁺ + 1) |
| 119 | S | (CH2)2CO2H | H | H | H | NHCH2(2,4-F2-Ph) | FAB-MS m/z:626(M⁺ + 1) |
| 120 | S | (CH2)2CO2H | H | H | H | NHCH2(3-Cl-Ph) | FAB-MS m/z:625(M⁺ + 1) |
| 121 | S | (CH2)2CO2H | H | H | H | NHCH2(3-MeO)-Ph) | FAB-MS m/z:620(M⁺ + 1) |
| 122 | S | (CH2)2CO2H | H | H | H | NHCH2(2-Py) | FAB-MS m/z:591(M⁺ + 1) |
| 123 | S | (CH2)2CO2H | H | H | H | 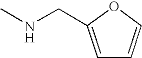 | FAB-MS m/z:580(M⁺ + 1) |
| 124 | S | (CH2)2CO2H | H | H | H | 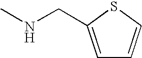 | FAB-MS m/z:596(M⁺ + 1) |
| 125 | S | (CH2)2CO2H | H | H | H | 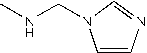 | FAB-MS m/z:608(M⁺ + 1) |
| 126 | S | (CH2)2CO2H | H | H | H | 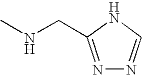 | FAB-MS m/z:581(M⁺ + 1) |
| 127 | S | (CH2)2CO2H | H | H | H | 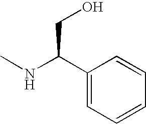 | FAB-MS m/z:620(M⁺ + 1) |
| 128 | S | (CH2)2CO2H | H | H | H | 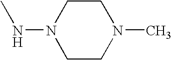 | FAB-MS m/z:598(M⁺ + 1) |
| 129 | S | (CH2)2CO2H | H | H | H | 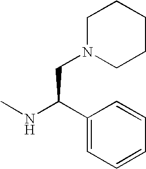 | FAB-MS m/z:687(M⁺ + 1) |
| 130 | S | (CH2)2CO2H | H | H | H | NH(CH2)2Ph | FAB-MS m/z:604(M⁺ + 1) |
| 131 | S | (CH2)2CO2H | H | H | H | NH(CH2)2OMe | FAB-MS m/z:558(M⁺ + 1) |
| 132 | S | (CH2)2CO2H | H | H | H | 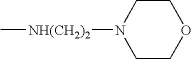 | FAB-MS m/z:613(M⁺ + 1) |
| 133 | S | (CH2)2CO2H | H | H | H | 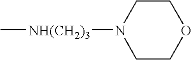 | FAB-MS m/z:627(M⁺ + 1) |
| 134 | S | (CH2)2CO2H | H | H | H | NH(CH2)2NHPh | FAB-MS m/z:619(M⁺ + 1) |
| 135 | S | (CH2)2CO2H | H | H | H | NH(CH2)2NH(2-Py) | FAB-MS m/z:620(M⁺ + 1) |
| 136 | S | (CH2)2CO2H | H | H | H | NH(CH2)3OMe | FAB-MS m/z:572(M⁺ + 1) |
| 137 | S | (CH2)2CO2H | H | H | H | NH(CH2)3CH3 | FAB-MS m/z:556(M⁺ + 1) |
| 138 | S | (CH2)2CO2H | H | H | H | 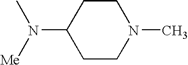 | FAB-MS m/z:611(M⁺ + 1) |
| 139 | S | (CH2)2CO2H | H | H | H | 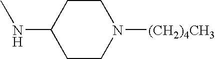 | FAB-MS m/z:653(M⁺ + 1) |
| 140 | S | (CH2)2CO2H | H | H | H | 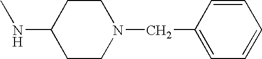 | FAB-MS m/z:673(M⁺ + 1) |
| 141 | S | (CH2)2CO2H | H | H | H | NEt2 | FAB-MS m/z:556(M⁺ + 1) |
| 142 | S | (CH2)2CO2H | H | H | H | 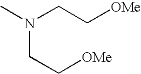 | FAB-MS m/z:616(M⁺ + 1) |
| 143 | S | (CH2)2CO2H | H | H | H | 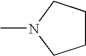 | FAB-MS m/z:554(M⁺ + 1) |
| 144 | S | (CH2)2CO2H | H | H | H | 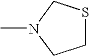 | FAB-MS m/z:572(M⁺ + 1) |
| 145 | S | (CH2)2CO2H | H | H | H | 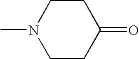 | FAB-MS m/z:582(M⁺ + 1) |
| 146 | S | (CH2)2CO2H | H | H | H | 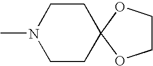 | FAB-MS m/z:626(M⁺ + 1) |
| 147 | S | (CH2)2CO2H | H | H | H | 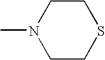 | FAB-MS m/z:586(M⁺ + 1) |
| 148 | S | (CH2)2CO2H | H | H | H | 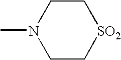 | FAB-MS m/z:618(M⁺ + 1) |
| 149 | S | (CH2)2CO2H | H | H | H | 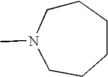 | FAB-MS m/z:582(M⁺ + 1) |
| 150 | S | (CH2)2CO2H | H | H | H | 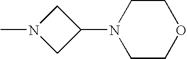 | FAB-MS m/z:625(M⁺ + 1) |
| 151 | S | (CH2)2CO2H | H | H | H | 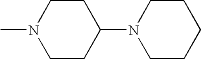 | FAB-MS m/z:651(M⁺ + 1) |
| 152 | S | (CH2)2CO2H | H | H | H | 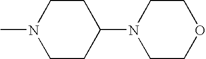 | FAB-MS m/z:653(M⁺ + 1) |
| 153 | S | (CH2)2CO2H | H | H | H | 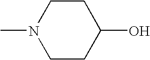 | FAB-MS m/z:584(M⁺ + 1) |
| 154 | S | (CH2)2CO2H | H | H | H | 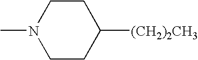 | FAB-MS m/z:610(M⁺ + 1) |
| 155 | S | (CH2)2CO2H | H | H | H | 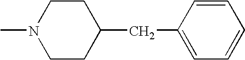 | FAB-MS m/z:658(M⁺ + 1) |
| 156 | S | (CH2)2CO2H | H | H | H | 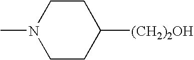 | FAB-MS m/z:612(M⁺ + 1) |
| 157 | S | (CH2)2CO2H | H | H | H | 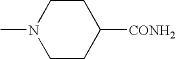 | FAB-MS m/z:611(M⁺ + 1) |
| 158 | S | (CH2)2CO2H | H | H | H | 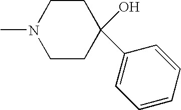 | FAB-MS m/z:660(M⁺ + 1) |
| 159 | S | (CH2)2CO2H | H | H | H | 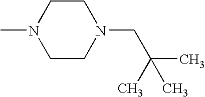 | FAB-MS m/z:639(M⁺ + 1) |
| 160 | S | (CH2)2CO2H | H | H | H | 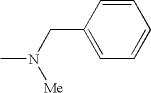 | FAB-MS m/z:604(M⁺ + 1) |
| 161 | S | (CH2)2CO2H | H | H | H | 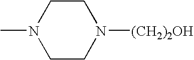 | FAB-MS m/z:613(M⁺ + 1) |
| 162 | S | (CH2)2CO2H | H | H | H | 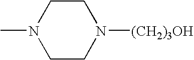 | FAB-MS m/z:627(M⁺ + 1) |
| 163 | S | (CH2)2CO2H | H | H | H | 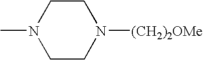 | FAB-MS m/z:627(M⁺ + 1) |
| 164 | S | (CH2)2CO2H | H | H | H | 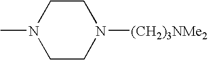 | FAB-MS m/z:654(M⁺ + 1) |
| 165 | S | (CH2)2CO2H | H | H | H | 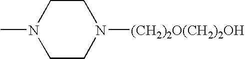 | FAB-MS m/z:657(M⁺ + 1) |
| 166 | S | (CH2)2CO2H | H | H | H | 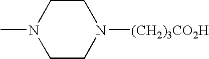 | FAB-MS m/z:656(M⁺ + 1) |
| 167 | S | (CH2)2CO2H | H | H | H | 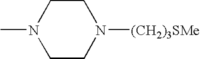 | FAB-MS m/z:657(M⁺ + 1) |
| 168 | S | (CH2)2CO2H | H | H | H | 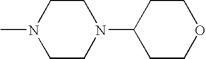 | FAB-MS m/z:653(M⁺ + 1) |
| 169 | S | (CH2)2CO2H | H | H | H | 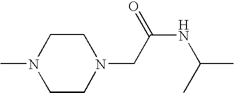 | FAB-MS m/z:668(M⁺ + 1) |
| 170 | S | (CH2)2CO2H | H | H | H | 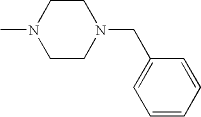 | FAB-MS m/z:659(M⁺ + 1) |
| 171 | S | (CH2)2CO2H | H | H | H | 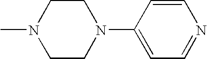 | FAB-MS m/z:646(M⁺ + 1) |
| 172 | S | (CH2)2CO2H | H | H | H | 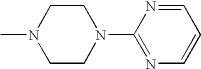 | FAB-MS m/z:647(M⁺ + 1) |
| 173 | S | (CH2)2CO2H | H | H | H | 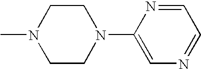 | FAB-MS m/z:647(M⁺ + 1) |
| 174 | S | (CH2)2CO2H | H | H | H | 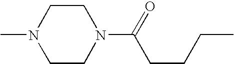 | FAB-MS m/z:653(M⁺ + 1) |
| 175 | S | (CH2)2CO2H | H | H | H | 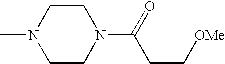 | FAB-MS m/z:655(M⁺ + 1) |
| 176 | S | (CH2)2CO2H | H | H | H | 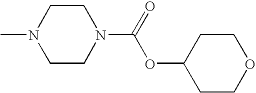 | FAB-MS m/z:697(M⁺ + 1) |
| 177 | S | (CH2)2CO2H | H | H | H | 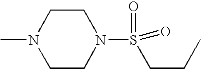 | FAB-MS m/z:675(M⁺ + 1) |
| 178 | S | (CH2)2CO2H | H | H | H | 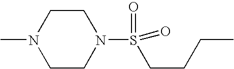 | FAB-MS m/z:689(M⁺ + 1) |
| 179 | S | (CH2)2CO2H | H | H | H | 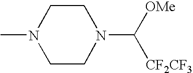 | FAB-MS m/z:701(M⁺ + 1) |
| 180 | S | (CH2)2CO2H | H | H | H | 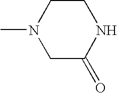 | FAB-MS m/z:583(M⁺ + 1) |
| 181 | S | (CH2)2CO2H | H | H | H | 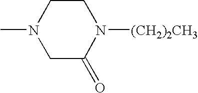 | FAB-MS m/z:625(M⁺ + 1) |
| 182 | S | (CH2)2CO2H | H | H | H | 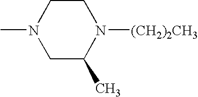 | FAB-MS m/z:625(M⁺ + 1) |
| 183 | S | (CH2)2CO2H | H | H | H | 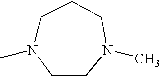 | FAB-MS m/z:597(M⁺ + 1) |
| 184 | S | (CH2)2CO2H | H | H | H | 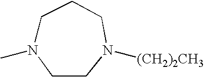 | FAB-MS m/z:625(M⁺ + 1) |
| 185 | S | (CH2)2CO2H | H | H | H | 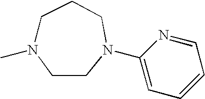 | FAB-MS m/z:660(M⁺ + 1) |
| 186 | S | (CH2)2CO2H | H | H | H | 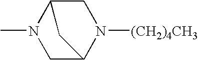 | FAB-MS m/z:651(M⁺ + 1) |

Of the compounds of the Examples mentioned above, those not specifically indicated were synthesized as free bases or free acids. In this connection, the compounds of the following Examples were synthesized as HCl or fumaric acid salts.

(HCl salts): Examples 3, 4, 7, 8, 10, 22, 27, 32, 35, 75, 84, 88, 89, 90, 91, 92, 93, 94, 95, 96, 97, 101, 102, 103, 107, 108, 109, 110, 111, 113, 119, 120, 123, 124, 132, 133, 134, 136, 137, 138 and 185 (Fumarate): Example 99

The following compounds can also be synthesized by employing production methods similar to these Examples or general synthesis methods.

TABLE 9

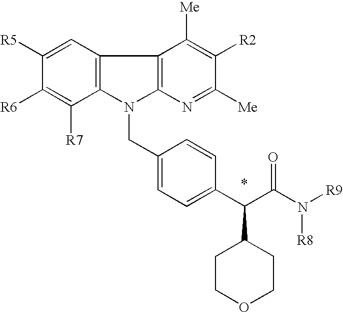

| | Configu- | | | | | |
|---|---|---|---|---|---|---|
| | ration | R2 | R5 | R6 | R7 | NR8R9 |
| 1 | S | CH2CO2H | H | H | H | N(CH3)2 |
| 2 | S | CH2CO2H | H | H | H | 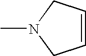 |
| 3 | S | CH2CO2H | H | H | H | 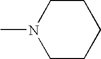 |
| 4 | S | CH2CO2H | H | H | H | 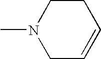 |
| 5 | S | CH2CO2H | H | H | H | 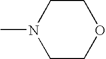 |
| 6 | S | CH2CO2H | H | H | H | 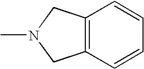 |
| 7 | S | CH2CO2H | H | H | H | 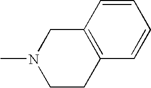 |
| 8 | S | CH2CO2H | H | H | H | 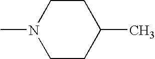 |
| 9 | S | CH2CO2H | H | H | H | 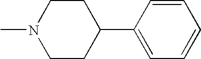 |
| 10 | S | CH2CO2H | H | H | H | 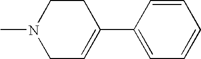 |
| 11 | S | CH2CO2H | H | H | H | 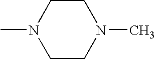 |
| 12 | S | CH2CO2H | H | H | H | 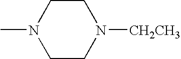 |
| 13 | S | CH2CO2H | H | H | H | 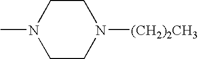 |
| 14 | S | CH2CO2H | H | H | H | 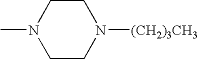 |
| 15 | S | CH2CO2H | H | H | H | 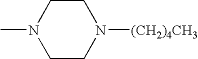 |
| 16 | S | CH2CO2H | H | H | H | 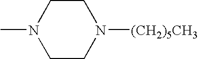 |
| 17 | S | CH2CO2H | H | H | H | 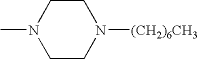 |
| 18 | S | CH2CO2H | H | H | H | 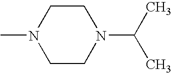 |
| 19 | S | CH2CO2H | H | H | H | 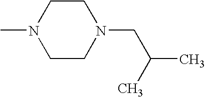 |
| 20 | S | CH2CO2H | H | H | H | 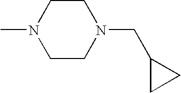 |
| 21 | S | CH2CO2H | H | H | H | 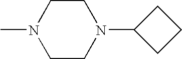 |
| 22 | S | CH2CO2H | H | H | H | 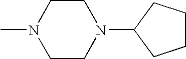 |
| 23 | S | CH2CO2H | H | H | H | 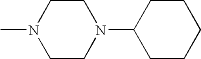 |
| 24 | S | CH2CO2H | H | H | H | 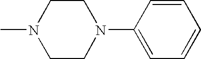 |
| 25 | S | CH2CO2H | H | H | H | 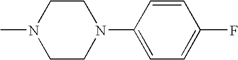 |
| 26 | S | CH2CO2H | H | H | H | 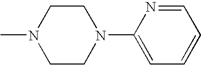 |
| 27 | S | CH2CCO2H | H | H | H | 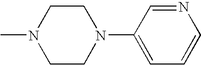 |
| 28 | S | CH2CO2H | H | H | H | 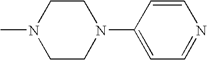 |
| 29 | S | CH2CO2H | H | H | H | 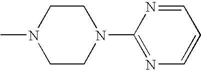 |
| 30 | S | CH2CO2H | H | H | H | 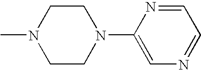 |
| 31 | S | (CH2)3CO2H | H | H | H | N(CH3)2 |
| 32 | S | (CH2)3CO2H | H | H | H | 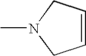 |
| 33 | S | (CH2)3CO2H | H | H | H | 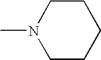 |
| 34 | S | (CH2)3CO2H | H | H | H | 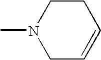 |
| 35 | S | (CH2)3CO2H | H | H | H | 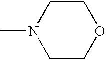 |
| 36 | S | (CH2)3CO2H | H | H | H | 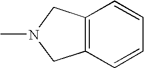 |
| 37 | S | (CH2)3CO2H | H | H | H | 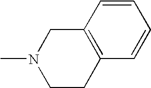 |
| 38 | S | (CH2)3CO2H | H | H | H | 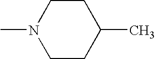 |
| 39 | S | (CH2)3CO2H | H | H | H | 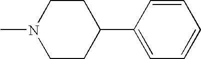 |
| 40 | S | (CH2)3CO2H | H | H | H | 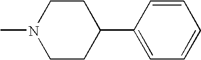 |
| 41 | S | (CH2)3CO2H | H | H | H | 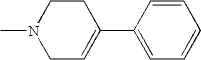 |
| 42 | S | (CH2)3CO2H | H | H | H | 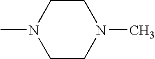 |
| 43 | S | (CH2)3CO2H | H | H | H | 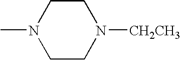 |
| 44 | S | (CH2)3CO2H | H | H | H | 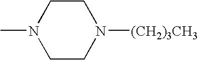 |
| 45 | S | (CH2)3CO2H | H | H | H | 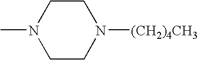 |
| 46 | S | (CH2)3CO2H | H | H | H | 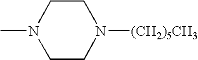 |
| 47 | S | (CH2)3CO2H | H | H | H | 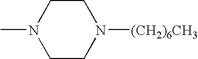 |
| 48 | S | (CH2)3CO2H | H | H | H | 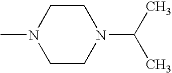 |
| 49 | S | (CH2)3CO2H | H | H | H | 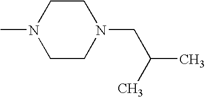 |
| 50 | S | (CH2)3CO2H | H | H | H | 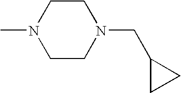 |
| 51 | S | (CH2)3CO2H | H | H | H | 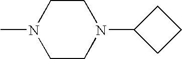 |
| 52 | 5 | (CH2)3CO2H | H | H | H | 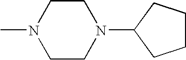 |
| 53 | S | (CH2)3CO2H | H | H | H | 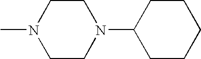 |
| 54 | S | (CH2)3CO2H | H | H | H | 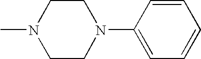 |
| 55 | 5 | (CH2)3CO2H | H | H | H | 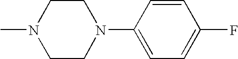 |
| 56 | S | (CH2)3CO2H | H | H | H | 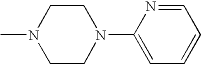 |
| 57 | S | (CH2)3CO2H | H | H | H | 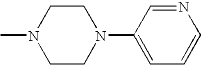 |
| 58 | S | (CH2)3CO2H | H | H | H | 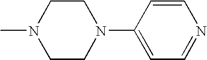 |
| 59 | S | (CH2)3CO2H | H | H | H | 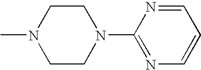 |
| 60 | S | (CH2)3CO2H | H | H | H | 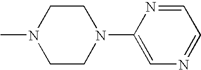 |
| 61 | 5 | CH2)2CO2H | H | H | F | N(CH3)2 |
| 62 | 5 | (CH2)2CO2H | H | F | H | N(CH3)2 |
| 63 | S | (CH2)2CO2H | F | H | H | N(CH3)2 |
| 64 | S | (CH2)2CO2H | H | H | F | 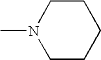 |
| 65 | S | (CH2)2CO2H | H | F | H | 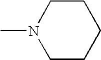 |
| 66 | S | (CH2)2CO2H | F | H | H | 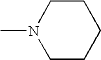 |
| 67 | S | (CH2)2CO2H | H | H | F | 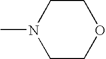 |
| 68 | S | (CH2)2CO2H | H | F | H | 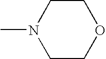 |
| 69 | S | (CH2)2CO2H | F | H | H | 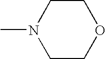 |
| 70 | S | (CH2)2CO2H | H | H | F | 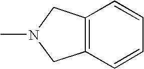 |
| 71 | S | (CH2)2CO2H | H | F | H | 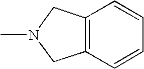 |
| 72 | S | (CH2)2CO2H | F | H | H | 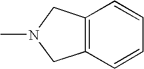 |
| 73 | S | (CH2)2CO2H | H | H | F | 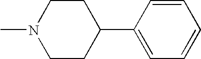 |
| 74 | S | (CH2)2CO2H | H | F | H | 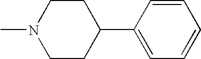 |
| 75 | S | (CH2)2CO2H | F | H | H | 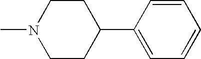 |
| 76 | S | (CH2)2CO2H | H | H | F | 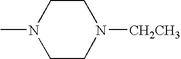 |
| 77 | S | (CH2)2CO2H | H | F | H | 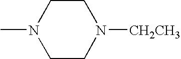 |
| 78 | S | (CH2)2CO2H | F | H | H | 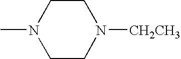 |
| 79 | S | (CH2)2CO2H | H | H | F | 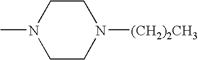 |
| 80 | S | (CH2)2CO2H | H | F | H | 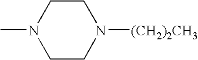 |
| 81 | S | (CH2)2CO2H | F | H | H | 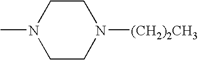 |
| 82 | S | (CH2)2CO2H | H | H | F | 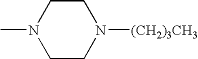 |
| 83 | S | (CH2)2CO2H | H | F | H | 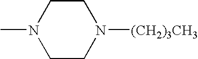 |
| 84 | S | (CH2)2CO2H | F | H | H | 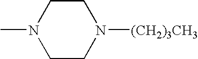 |
| 85 | S | (CH2)2CO2H | H | H | F | 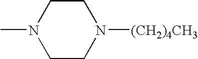 |
| 86 | S | (CH2)2CO2H | H | F | H | 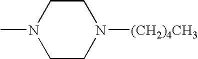 |
| 87 | S | (CH2)2CO2H | F | H | H | 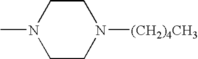 |
| 88 | S | (CH2)2CO2H | H | H | F | 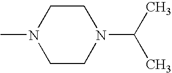 |
| 89 | S | (CH2)2CO2H | H | F | H | 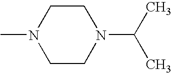 |
| 90 | S | (CH2)2CO2H | F | H | H | 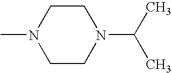 |
| 91 | S | (CH2)2CO2H | H | H | F | 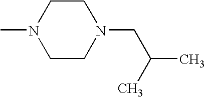 |
| 92 | S | (CH2)2CO2H | H | F | H | 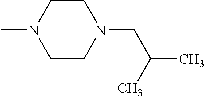 |
| 93 | S | (CH2)2CO2H | F | H | H | 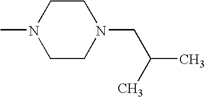 |
| 94 | S | (CH2)2CO2H | H | H | F | 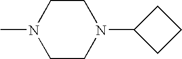 |
| 95 | S | (CH2)2CO2H | H | F | H | 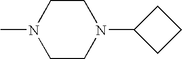 |
| 96 | S | (CH2)2CO2H | F | H | H | 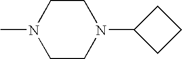 |
| 97 | S | (CH2)2CO2H | H | H | F | 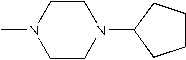 |
| 98 | S | (CH2)2CO2H | H | F | H | 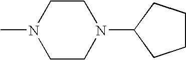 |
| 99 | S | (CH2)2CO2H | F | H | H | 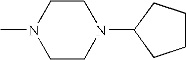 |
| 100 | S | (CH2)2CO2H | H | H | F | 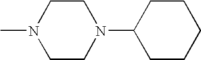 |
| 101 | S | (CH2)2CO2H | H | F | H | 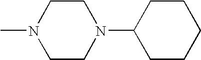 |
| 102 | S | (CH2)2CO2H | F | H | H | 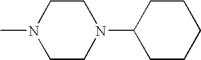 |
| 103 | S | (CH2)2CO2H | H | H | F | 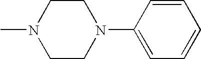 |
| 104 | S | (CH2)2CO2H | H | F | H | 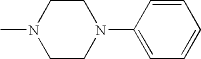 |
| 105 | S | (CH2)2CO2H | F | H | H | 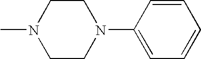 |
| 106 | S | (CH2)2CO2H | H | H | F | 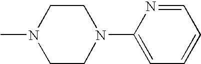 |
| 107 | S | (CH2)2CO2H | H | F | H | 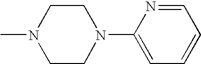 |
| 108 | S | (CH2)2CO2H | F | H | H | 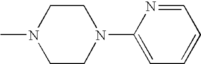 |

(Test Methods)

The apo B-related lipoprotein secretion-inhibiting activity and blood cholesterol and triglyceride lowering effect in rats of the compounds of the invention were verified by the following test methods.

1. Measurement of Apo B-Related Lipoprotein Secretion in Liver Cells

Hep G2 cells were cultured in a 96-well plate using a 10% fetal bovine serum-containing Dulbecco's modified Eagle's medium (to be referred to as “10% FBS-containing DMEM” hereinafter). The medium was exchanged with a 10% FBS-containing DMEM that contained a DMSO solution of the test compound (DMSO final concentration 0.1%), and the cells were further incubated therein for 18 hours to obtain the culture supernatant. The culture supernatant obtained from a 10% FBS-containing DMEM that contained DMSO alone (final concentration 0.1%) was used as the control. The apo B-related lipoprotein produced in the culture supernatant was determined through an enzyme immunoassay in the manner mentioned below.

A solution of anti-human apolipoprotein B monoclonal antibody (4 μg/ml) was dispensed in 100 μl portions into a 96-well immunoassay plate and kept at 4° C. for 18 hours. Each well was washed three times with a phosphate buffer to which had been added 0.1% Tween 20 (to be referred to as “PBS-T” hereinafter), and 300 μl of a 4-fold diluted immune blocker “Block Ace” was added thereto and kept at 37° C. for 1 hour. The aforementioned culture supernatant was added to it, and kept at 37° C. for 3 hours. This was washed three times with PBS-T, and then 100 μl of a solution of caprine anti-human apolipoprotein B polyclonal antibody (2,000-fold dilution) was added to it and kept at 37° C. for 1 hour. This was washed three times with PBS-T, and then 100 μl of a solution of alkaline phosphatase-labeled anti-caprine IgG antibody (2,000-fold dilution) was added thereto and kept at 37° C. for 1 hour. This was washed four times with PBS-T, once with PBS and then with a carbonate buffer (pH 9.5), and 100 μl of a solution of an alkaline phosphatase substrate was added thereto and kept at room temperature for 5 minutes. A 50 μl portion of 0.2 M sodium hydroxide was added to it to stop the reaction, and the absorbance at 405 nm was measured. From the calibration curve based on a standard, human low-density lipoprotein, the absolute amount of apo B-related lipoprotein was obtained.

The result is shown in he following table (Table 10).

TABLE 10

| Ex. | IC50(nM) |
|---|---|
| 17 | 4.8 |
| 18 | 3.0 |
| 19 | 1.7 |
| 37 | 29 |
| 40 | 12 |
| 41 | 40 |
| 42 | 3.8 |
| 45 | 28 |
| 49 | 4.9 |
| 50 | 3.2 |
| 54 | 2.2 |
| 55 | 11 |
| 56 | 7.2 |
| 57 | 8.0 |
| 58 | 2.1 |
| 60 | 1.7 |
| 61 | 3.3 |
| 117 | 2.2 |

It was shown that the compounds of the invention inhibit secretion of apo B-related lipoprotein.

2. Measurement of Blood Cholesterol and Triglyceride in Rats

The influence of test compounds on VLDL secretion in vivo in rats was investigated. A 0.5% methyl cellulose suspension containing a test compound was orally administered to male SD rats. After 1 hour, a physiological saline solution of Triton WR-1339 (corresponding to 400 mg/kg) was injected through the caudal vein. After 4 hours, their blood was collected under anesthetization with diethyl ether. The total cholesterol and triglyceride in blood were measured by the use of “Cholesterol C Test Wako” and “Triglyceride G Test Wako” (both from Wako Pure Chemical Industries), respectively. The rats orally administered with the 0.5% methyl cellulose solution and then intravenously injected with physiological saline were used as a normal group, and those orally administered with the 0.5% methyl cellulose solution and then intravenously injected with physiological saline of Triton WR-1339 were used as a control group. The difference in the total cholesterol (TC) or triglyceride (TG) in the serum between the control group and the normal group was defined as the amount of secretion per hour, and the lowering effect in the amount of secretion of the test compound was represented by ED₅₀.

The result is given in the following table (Table 11).

TABLE 11

ED50(mg/kg)

| Ex. | TC | TG |
|---|---|---|
| 17 | 0.74 | 0.39 |
| 18 | 0.50 | 0.52 |
| 19 | 0.17 | 0.24 |
| 23 | 0.27 | 0.19 |
| 24 | 0.21 | 0.22 |
| 37 | 0.71 | 0.71 |
| 40 | 0.47 | 0.28 |
| 41 | 0.68 | 0.77 |
| 42 | 0.39 | 0.38 |
| 49 | 0.86 | 0.80 |
| 50 | 0.48 | 0.41 |
| 54 | 0.44 | 0.41 |
| 55 | 0.35 | 0.36 |
| 56 | 0.9 | 1.3 |
| 57 | 0.74 | 0.56 |
| 58 | 1.9 | 1.1 |
| 60 | 0.23 | 0.14 |
| Implitapide | 1.0 | 1.1 |

Implitapide: Compound of Example 5 in the JP-A 8-225526

The cholesterol and triglyceride-lowering effect of the compounds of the invention is comparative to or higher than that of Implitapide having the same efficacy.

3. Pharmacological Test on High-Cholesterol Feed-Loaded Rats

The influence of test compounds on plasma lipid in vivo was investigated with high-cholesterol feed-loaded rats. A 0.5% methyl cellulose suspension containing a test compound was forcedly orally administered to male SD rats loaded with high-cholesterol feed (1.5% cholesterol, 10% coconut oil, 0.5% cholic acid), once a day for continuous 7 days. After the final administration, the rats were fed with nothing, and after 6 hours, their blood was collected under anesthetization with diethyl ether. Using an automatic analyzer, Hitachi's Model 7250, the total cholesterol, HDL cholesterol and triglyceride in plasma were measured. Non-HDL cholesterol was obtained by subtracting HDL cholesterol from total cholesterol. The non-HDL cholesterol lowering effect of the test compound, relative to the control group, was represented by ED₅₀.

The result is given in the following table (Table 12).

TABLE 12

| Ex. | ED50 (mg/kg) | Ex. | ED50 (mg/kg) |
|---|---|---|---|
| 17 | 0.14 | 49 | 0.43 |
| 18 | 0.20 | 50 | 0.19 |
| 19 | 0.059 | 54 | 0.14 |
| 23 | 0.063 | 55 | 0.28 |
| 24 | 0.040 | 56 | 1.1 |
| 40 | <0.1 | 57 | 0.88 |
| 41 | 0.21 | 60 | 0.045 |
| 42 | 0.10 | 61 | 0.040 |
| 45 | 0.41 | 117 | 0.032 |
| Implitapide | 1.0 | | |

It was confirmed that the compounds of the invention have an excellent cholesterol lowering effect in dietetic hypercholesterolemia and their activity is comparative to or at least 30 times higher than that of Implitapide, and they are useful therefore as a cholesterol lowering agent.
4. Pharmacological test in KK-Ay Mice

The influence of test compounds on plasma lipid in vivo was investigated with KK-Ay mice. A 0.5% methyl cellulose suspension containing a test compound was forcedly orally administered to male KK-Ay mice once a day for continuos 7 days. After the final administration, the mice were fed with nothing, and after 6 hours, their blood was collected under anesthetization with diethyl ether. Using the automatic analyzer, Hitachi's Model 7250, the total cholesterol, HDL cholesterol and triglyceride in plasma were measured. Non-HDL cholesterol was obtained by subtracting HDL cholesterol from total cholesterol. The plasma lipid lowering effect of the test compound was expressed in percentage relative to the control group.

The result is given in the following table (Table 13).

TABLE 13

Lowering rate
(dose, 3 mg/kg)

| Ex. | non-HDL | TG |
|---|---|---|
| 17 | 85% | 86% |
| 18 | 85% | 87% |
| 19 | 95% | 91% |
| 23 | 91% | 96% |
| 24 | 96% | 95% |
| 37 | 62% | 57% |
| 40 | 74% | 81% |
| 41 | 63% | 66% |
| 42 | 84% | 91% |
| 45 | 86% | 92% |
| Implitapide | 14% | 15% |
| | (10 mg/kg) | (10 mg/kg) |
| 49 | 83% | 87% |
| 50 | 90% | 94% |
| 54 | 80% | 85% |
| 55 | 82% | 86% |
| 56 | 63% | 41% |
| 57 | 86% | 89% |
| 58 | 80% | 83% |
| 60 | 97% | 98% |
| 61 | 96% | 98% |
| 117 | 98% | 98% |

It was confirmed that the compounds of the invention have an cholesterol and triglyceride lowering effect superior to that of Implitapide, even in disease models having high cholesterol and high triglyceride, suggesting their usefulness as a medicament for hyperlipemia.

### INDUSTRIAL APPLICABILITY

According to the invention, the compounds of the invention which show apo B-related lipoprotein secretion-inhibiting activity have excellent blood cholesterol and triglyceride lowering effect, and they are useful as remedies for hyperlipemia, arteriosclerosis, obesity and pancreatitis, through their single administration or by their combination with other lipid lowering agents.

1. A tetrahydropyran derivative represented by the following general formula (I) or a salt thereof 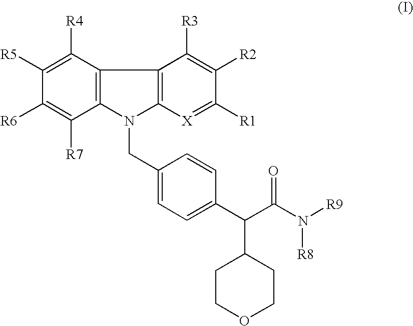
(symbols in the formula mean as follows;
R¹ and R³: the same or different and each represents H or lower alkyl,
R²: H, halogen, R^{a}-lower alkyl-, or R²⁰O—CO—,
R^{a}: H, R²¹O—CO—, R²²R²³N—, R²⁴R²N—CO—, R²⁶O—, cyano, or optionally-substituted hetero ring-,
R⁴, R⁵, R⁶ and R⁷: the same or different and each represents H, halogen, haloalkyl, cyano, lower alkyl, lower alkyl-O—, R²¹O—CO-lower alkyl-, R²⁷—CO—, or R²R²⁹N—S(O)₂
R⁸ and R⁹: the same or different and each represents H, lower alkyl, R³⁰-lower alkyl-, R³¹R³²N—, optionally-substituted hetero ring-, or R³³R³⁴R³⁵C—, with the proviso that R⁸ and R⁹ may together form a optionally-substituted hetero ring
X: N, or CR³⁶,
R²⁰, R²² to R²⁶, R²⁸, R²⁹, R³², R³⁵ and R³⁶: the same or different and each represents H, or lower alkyl,
R²¹: H, lower alkyl, or aryl-lower alkyl-,
R²⁷: HO—, lower alkyl-O—, or optionally-substituted hetero ring-,
R³⁰: optionally-substituted aryl, optionally-substituted hetero ring-, or lower alkyl-O—,
R³¹: optionally-substituted aryl, or optionally-substituted hetero ring-,
R³³: HO-lower alkyl-, or optionally-substituted hetero ring-lower alkyl-, and
R³⁴: optionally-substituted aryl).

2. The tetrahydropyran derivative or salt thereof according to claim 1, wherein the optionally-substituted aryl and optionally-substituted hetero ring in the aforementioned general formula (1) may be substituted with 1 to 3 substituents selected from the group shown below; substituents: halogen, oxo, lower alkyl optionally substituted with OH, haloalkyl-optionally substituted with lower alkyl-O—, lower alkenyl, C₃₋₈ cycloalkyl, C₃₋₈ cycloalkyl-lower alkyl-, aryl optionally substituted with halogen atom(s), aryl-lower alkyl-, heterocyclic group, HO—, lower alkyl-O—, lower alkyl-O-lower alkyl optionally substituted with OH, lower alkyl-O-lower alkyl-CO—, lower alkyl-S(O)ₘ—, lower alkyl-S(O)ₘ-lower alkyl-, lower alkyl-O—CO—, HO—CO-lower alkyl-, HO—CO-lower alkyl-O—, R^{x}R^{y}N—CO—, R^{x}R^{y}N—CO-lower alkyl-, R^{x}R^{y}N-lower alkyl-, lower alkyl-CO—, aryl-lower alkyl-O—CO— and hetero ring-O—CO-(symbols in the formulae means as follows;
R^{x}and R^{y}: the same or different and each represents H or lower alkyl,
m: 0, 1 or 2).

3. The tetrahydropyran derivative or salt thereof according to claim 1, wherein, in the aforementioned general formula (I), R² is R^{a}-lower alkyl-, R⁴, R⁵, R⁶ and R⁷ may be the same or different and each represents H or halogen, R⁸ and R⁹ may be the same or different and each represents H, lower alkyl or R³⁰-lower alkyl-, or R⁸ and R⁹ may together form an optionally-substituted hetero ring, and X is N.

4. A compound selected from 3-(2,4-dimethyl-9-{4-[(S)-2-oxo-2-(4-pyridin-2-ylpiperazin-1-yl)-1-(tetrahydro-2H-pyran-4-yl)ethyl]benzyl}-9H-pyrido[2,3-b]indol-3-yl)propanoic acid; 3-(2,4-dimethyl-9-{4-[(S)-2-oxo-2-(4-propylpiperazin-1-yl)-1-(tetrahydro-2H-pyran-4-yl)ethyl]benzyl}-9H-pyrido[2,3-b]indol-3-yl)propanoic acid; 3-(9-{4-[(S)-(benzylcarbamoyl)(tetrahydro-2H-pyran-4-yl)methyl]benzyl}-2,4-dimethyl-9H-pyrido[2,3-b]indol-3-yl)propanoic acid; 3-(2,4-dimethyl-9-{4-[(S)-(dimethylcarbamoyl)(tetrahydro-2H-pyran-4-yl)methyl]benzyl}-9H-pyrido[2,3-b]indol-3-yl)propanoic acid; 3-(2,4-dimethyl-9-{4-[(S)-2-morpholin-4-yl-2-oxo-1-(tetrahydro-2H-pyran-4-yl)ethyl]benzyl}-9H-pyrido[2,3-b]indol-3-yl)propanoic acid; 3-(2,4-dimethyl-9-{4-[(S)-2-(4-isobutylpiperazin-1-yl)-2-oxo-1-(tetrahydro-2H-pyran-4-yl)ethyl]benzyl}-9H-pyrido[2,3-b]indol-3-yl)propanoic acid; 3-9-{4-[(S)-2-(4-cyclobutylpiperazin-1-yl)-2-oxo-1-(tetrahydro-2H-pyran-4-yl)ethyl]benzyl}-2,4-dimethyl-9H-pyrido[2,3-b]indol-3-yl)propanoic acid; 3-9-{4-[(S)-2-(4-cyclopentylpiperazin-1-yl)-2-oxo-1-(tetrahydro-2H-pyran-4-yl)ethyl]benzyl}-2,4-dimethyl-9H-pyrido[2,3-b]indol-3-yl)propanoic acid; and their salts.

5. A pharmaceutical composition which comprises the tetrahydropyran derivative described in claim 1 or a pharmaceutically acceptable salt thereof.

6. A method for preventing or treating hyperlipemia, which comprises administering a therapeutically effective amount of the tetrahydropyran derivative described in claim 1 or a pharmaceutically acceptable salt thereof and a pharmaceutically acceptable carrier to a patient who requires the treatment.

7. A therapeutic agent for hyperlipemia, characterized in that the tetrahydropyran derivative described in claim 1 or a pharmaceutically acceptable salt thereof is administered in combination with one or two or more of lipid lowering agents.

8. A method for preventing or treating hyperlipemia, by administering a therapeutically effective amount of the tetrahydropyran derivative described in claim 1 or a pharmaceutically acceptable salt thereof and a pharmaceutically acceptable carrier, simultaneously or separately with one or two or more of lipid lowering agents, to a patient who requires the treatment.

9. A kit in which a pharmaceutical composition comprising the tetrahydropyran derivative described in claim 1 or a pharmaceutically acceptable salt thereof and a pharmaceutically acceptable carrier, and a lipid lowering agent, are each independently packaged.
